(19) United States
(12) Patent Application Publication
(10) Pub. No.: US 2020/0061212 A1
(43) Pub. Date: Feb. 27, 2020

(54) SOLID ORAL COMPOSITION CONTAINING DYES

(71) Applicant: COSMO TECHNOLOGIES LTD., Dublin 2 (IE)

(72) Inventor: Luigi MORO, Lainate (IT)

(73) Assignee: COSMO TECHNOLOGIES LTD., Dublin 2 (IE)

(21) Appl. No.: 16/463,320

(22) PCT Filed: Nov. 28, 2017
(86) PCT No.: PCT/EP2017/080574

§ 371 Date: May 22, 2019

Related U.S. Application Data
(60) Provisional application No. 62/426,903, filed on Nov. 28, 2016.
(60) Provisional application No. 62/587,109, filed on Nov. 16, 2017.

(51) Int. Cl. A61K 49/00; A61K 9/20; A61K 9/28

(52) U.S. Cl. CPC A61K 49/006; A61K 49/0089; A61K 9/2846; A61K 9/2054; A61K 9/2018

(57) ABSTRACT

The present invention relates to methods for improving the detection of pathologies in the colon and method of flagging the mucosal lesions in the colon.

### BACKGROUND

Endoscopy is an exceptionally important diagnostic technique for the diagnosis of inflammatory, ulcerative, and neoplastic pathologies of the gastrointestinal tract.

Actually, endoscopy allows observing—from inside the lumen—the state of preservation and development of the mucosa that covers the gastrointestinal cavity, as well as the surface spraying thereof, the presence of deformations, and/or neoformations, and/or ulcerations.

Increasingly more powerful and sophisticated endoscope probes have considerably improved this technique. The progress of the materials employed has also improved performance in terms of illumination technologies and resolution power.

More recently, there has been an improvement of the conventional diagnostic-therapeutic aspects involving image magnification and vital dyes, used to locally develop a contrasting colour capable of amplifying the resolution diagnostic power of the conventional technique. The use of dyes in diagnostic endoscopic procedures is described by “chromoendoscopy”, particularly useful for identifying suspicious areas displaying degenerative characteristics.

The use of colouring is generally adopted in the second part of the endoscopic analysis, during the step of withdrawing the endoscopic probe, and after accurately cleaning the mucosa tract to be examined. Currently, the dye is applied to the mucosa by spraying a certain volume of a dye-containing solution using a catheter or capillary pipe directly inserted into the working channel of the endoscopic probe.

The diffusion of the dye on the cell surface or the extent of absorption by the vital cells markedly differentiates the cells with normal vitality from those cells, such as neoplastice cells, in the advanced replication stage.

The dyes usually used are mainly, but not exclusively, the following: methylene blue, congo red, carmine indigo, and/or toluidine blue.

Methylene blue and toluidine blue are uniformly absorbed by the whole intestinal mucosa but that absorption is reduced in an inflammatory environment, particularly as the phlogosis, i.e, inflammation, worsens. Due to this characteristic, the two dyes are also useful to ascertain whether inflammatory processes are in remission, and are also useful in distinguishing between pseudopolyps and true polyps. Indeed, inflamed or malignant/premalignant colonic epithelium exhibits decreased cytoplasm and goblet cells that are either reduced in amount or absent. These alterations result in decreased uptake of methylene blue and endoscopic appearance of focal light blue or pink (unstained) or heterogeneously stained (specked) mucosa in contrast to a more uniform staining pattern when colonic mucosa is not affected by pathologic processes. Differently from this concept, carmine indigo is not absorbed by cells and functions as a contrast agent increasing visibility of mucosal structures and enhancing details of normal and abnormal colonic patterns. Carmine indigo thus finds application in long duration inflammatory forms and can be used to highlight flat lesions, which can contain tumoral forms, which are difficult to detect with conventional white light endoscopy that does not employ contrasting colours.

Within the dyeing procedure, it should be observed that use thereof reveals several practical problems that can be difficult to resolve due to the challenges involved in applying the dye. First and foremost the pharmacy of the institute where the endoscopy is performed should be capable of preparing solutions with concentrations of dye generally ranging from 0.1% to 1%; then the dye should be dispensed (using a dedicated spray catheter) uniformly so as to cover homogeneously the mucosal surface subject of the evaluation.

Furthermore, the sprayed dye excess is to be removed after a few minutes through washing and sucking operations. That removal of excess dye requires additional time after each repetition of the dyeing spray process during the colonoscopy. The process, consequently, is time consuming for both nurses and physicians and makes it difficult to maximize the efficiency of the schedule of endoscopic procedures. The procedure is sufficiently rare that it tends to be operator-dependent, requiring a dedicated learning curve to obtain the right level of expertise to be able to evaluate the specific staining patterns obtained and their significance.

The need for the simultaneous presence of these precise conditions contributes to the difficulty of executing the chromoendoscopy procedure. Those difficulties have resulted in the procedure being carried out by only a minority of endoscopy units in hospitals and nursing homes specialized in gastroenterology.

Furthermore, other problems have resulted. The conventional local spraying of a solution on the mucosal wall may fail to reveal forms that are latent but still too small to detect and may fail to reveal the degenerative processes of the digestive system.

Moreover, locally spraying a solution can result in a short performance time of the dye. In particular, the time between spraying of the dye and observation is generally only a few seconds or a couple of minutes, a period known to be too short for allowing a consistent absorption of the dye to provide good contrast development and also achievement of good staining efficacy. Those issues may make it difficult for the endoscopist to intervene to obtain good detection and evaluation, as for example, in a biopsy.

Furthermore, the experience of each endoscopist who performs the procedure is somewhat subjective, additionally generating problems in the execution of both the endoscopic and related diagnostic evaluations. As a practical difficulty, such subjectivity resulting from the experience and convenience of the operator can undesirably lead to great variability in results. And the experience of the endoscopist plays an important role: the more experienced endoscopist, compared to the less experienced endoscopist, may spot suspicious areas when the dye is sprayed according to the current chromoendoscopy, further exacerbating the subjectivity of the test results.

Significant variability in test results can also result from the apparatus used, as well as from the acceptability of a particular patient to the diagnostic evaluation practice.

Thus, there arises the need of providing further improvement in both simplicity and safety from use of a dye in diagnostic endoscopies. It is desirable to improve the means of administration to provide a homogeneous and complete distribution of the dye for an improved effect in evaluating a treated area.

And as will be evident from above, it is desirable to obtain improvements that will increase the objectivity of the endoscopic evaluation to allow an improved diagnostic evaluation.

Particularly, in the case of colonic endoscopy (colonoscopy), a need still exists for providing an improved mucosal staining and ameliorating the efficacy of the diagnostic endoscopy evaluation.

### SUMMARY OF THE INVENTION

It has been surprisingly discovered that a specific solid composition in the form of tablets containing at least one dye and at least one physiologically acceptable excipient, orally administered according to a defined schedule prior to endoscopy, can provide an improved mucosal staining and can ensure a proper interaction between the dye and the colonic mucosa, obtaining the flagging of the lesions, which are consequently differentiated from the surrounding healthy mucosa.

In some embodiments are provided any of the methods disclosed herein wherein a solid composition is administered to a human as an aid for detection and visualization of adenomas and carcinomas in humans undergoing colonoscopy. In any of the embodiments described herein, the term “bowel cleansing solution” means any aqueous preparation or solution that is consumed by the human, including ordinary tap or bottled water or an aqueous solution comprising other compounds described herein, including one or more of an osmotic laxative, sodium sulfate, potassium sulfate, magnesium sulfate, polyethylene glycol, sodium chloride, sodium bicarbonate, potassium chloride, potassium picosulfate, sodium picosulfate and flavorings.

In some embodiments are provided any of the methods disclosed herein wherein a solid composition is administered to a human to visualize colonic adenoma in patients undergoing screening colonoscopy, including patients at high risk of colorectal carcinoma (CRC), including those with previous history of polyps at prior colonoscopy, patients with colorectal cancer and patients with family history.

In one aspect, the present invention provides a method for improving the detection of pathologies in the colon, comprising orally administering to a human 4 liters of a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liters of bowel cleaning solution;
c) 2 unit dosages of the solid composition after the intake of 4^{th} liter of bowel cleaning solution;
   wherein each unit dosage of the solid composition contains 25 mg of methylene blue, whereby the adenoma detection rate is at least about 40%.

In one embodiment is provided a method for improving the detection of pathologies in the colon of a human, comprising orally administering to the human 8 tablets of a solid composition and a volume of a bowel cleaning solution, wherein the solid composition is administered orally in three doses during the intake of the bowel cleansing solution according to the following schedule: (a) a first dose comprising administration of 3 tablets of the solid composition to the human following consumption of at least one liter of bowel cleansing solution; (b) a second dose comprising administration of 3 tablets of the solid composition to the human about 1 hour following administration the first dose of the solid composition; and (c) a third dose comprising administration of 2 tablets of the solid composition to the human about 1 hour following administration of the second dose of the solid composition. In some embodiments, at least 1 total liter of bowel cleansing solution is consumed by the human in combination with the administration of the 8 tablets of the solid composition. In some embodiments, at least 2 liters of bowel cleansing solution is consumed by the human in combination with the administration of the 8 tablets of the solid composition. In some embodiments, at least 3 liters of bowel cleansing solution is consumed by the human in combination with the administration of the 8 tablets of the solid composition. In some embodiments, a total of 4 liters of bowel cleansing solution is consumed by the human in combination with the administration of the 8 tablets of the solid composition. In some embodiments, the entire volume of bowel cleansing solution is consumed by the human in combination with the 8 tablets of the solid composition at least 8 hours prior to an endoscopic procedure being performed on the human. In some embodiments, the human consumes one half or less of the total volume of bowel cleansing solution in combination with the administration of the 8 tablets of the solid composition the day before an endoscopic procedure is performed and consumes the remaining portion of the bowel preparation solution the day the endoscopic procedure is performed. In some embodiments, the entire volume of bowel preparation solution is consumed at least two hours prior to the endoscopic procedure. In some embodiments, the bowel cleansing solution is consumed by the human according to the schedule: (a) the day before the endoscopic procedure, the human consumes a volume of at least 16 ounces of bowel preparation solution, followed by the consumption of at least 32 ounces of water over the next hour, in combination with the administration of the 8 tablets of the solid composition; and (b) the day of the endoscopic procedure, the human consumes at least 16 ounces of bowel preparation solution, followed by the consumption of at least 32 ounces of water over the next hour.

In one embodiment is provided a method for improving the detection of pathologies in the colon of a human during an endoscopic procedure, comprising orally administering to the human 8 tablets of a solid composition and a volume of a bowel cleaning solution, wherein the solid composition is administered orally during the intake of the bowel cleansing solution according to the following schedule: (a) the day before the endoscopic procedure, the human consumes a volume of at least 16 ounces of bowel preparation solution, followed by the consumption of at least 32 ounces of water over the next hour; and (b) the day of the endoscopic procedure, the human consumes at least 16 ounces of bowel preparation solution, followed by the consumption of at least 32 ounces of water over the next hour. In some embodiments, all 8 tablets of the solid composition are administered to the human the day before the endoscopic procedure. In some embodiments, a portion of the 8 tablets of the solid composition are administered to the human the day before the endoscopic procedure, and the remaining tablets of the solid composition are administered to the human the day of the endoscopic procedure. In some embodiments, the entire volume of bowel preparation solution is consumed at least two hours prior to the endoscopic procedure. In some embodiments, the 8 tablets of the solid composition are administered to the human at least 8 hours prior to the endoscopic procedure.

In some embodiments are provided any of the disclosed methods wherein the human is administered 8 tablets of a solid composition and consumes a volume of a bowel cleansing solution, wherein the bowel cleaning is consumed according to the following schedule: (a) the day before the endoscopic procedure, the human consumes a volume of at least 16 ounces of bowel preparation solution, followed by the consumption of at least 32 ounces of water over the next hour; and (b) the day of the endoscopic procedure, the human consumes at least 16 ounces of bowel preparation solution, followed by the consumption of at least 32 ounces of water over the next hour. In some embodiments, the human has been administered all 8 tablets of the solid composition and consumed the entire volume of bowel cleansing solution at least 8 hours prior to the endoscopic procedure. In some embodiments, the human is administered all 8 tablets of the solid composition the day before the endoscopic procedure and consumes the entire volume of bowel cleansing solution at least 2 hours prior to, or up until 2 hours before, the endoscopic procedure. In some embodiments, the human is administered all 8 tablets of the solid composition at least 8 hours prior to the endoscopic procedure and consumes the entire volume of bowel cleansing solution at least 2 hours prior to, or up until 2 hours before, the endoscopic procedure.

In some embodiments are provided any of the disclosed methods wherein the human is administered 8 tablets of a solid composition and consumes a volume of a bowel cleansing solution, wherein a total volume of 4 liters of the bowel cleaning is consumed at a rate of 240 mL (8 ounces) every 10 minutes, until 4 liters are consumed or until rectal effluent is clear. In some embodiments are provide any of the disclosed methods, wherein the bowel cleansing solution is delivered to the human by nasogastric tube at a rate of from about 1.2 liters per hour to about 1.8 liters per hour. In some embodiments are provide any of the disclosed methods, wherein the human drinks a volume of bowel cleansing solution at a rate of 25 mL/kg/hour until 4 liters are consumed or until watery stool is clear and free of solid matter. In some embodiments, the human has been administered all 8 tablets of the solid composition and consumed the entire volume of bowel cleansing solution at least 8 hours prior to the endoscopic procedure. In some embodiments, the human has been administered all 8 tablets of the solid composition after at least one liter of the bowel cleansing solution the day before the endoscopic procedure, and completed the intake of the entire volume of bowel cleansing solution at least 2 hours prior to, or up until 2 hours before, the endoscopic procedure. In some embodiments, the human has been administered all 8 tablets of the solid composition after at least one liter of the bowel cleansing solution at least 8 hours prior to the endoscopic procedure and completed the intake of the entire volume of bowel cleansing solution at least 2 hours prior to, or up until 2 hours before, the endoscopic procedure.

In some embodiments, the bowel cleansing solution comprises one or more of an osmotic laxative, sodium sulfate, potassium sulfate, magnesium sulfate, polyethylene glycol, sodium chloride, sodium bicarbonate, potassium chloride, potassium picosulfate, sodium picosulfate and flavorings. In some embodiments, the bowel cleansing solutions comprises polyethylene glycol, such as polyethylene glycol 3350, sodium bicarbonate, sodium chloride, and potassium chloride. In some embodiments, the bowel cleansing solution does not contain phosphate. In some embodiments, the bowel cleansing solution does not produce any clinically significant electrolyte shifts in the human upon consumption by the human. In some embodiments, the bowel cleansing solution may comprise phosphate in an amount that does not produce any clinically significant electrolyte shifts in the human upon consumption by the human. In some embodiments, the bowel cleansing solution is in the form of an oral solution for dilution. In some embodiments, the bowel cleansing solution is prepared by dissolution of a powder with water or a composition comprising water, such as an electrolyte solution. In some embodiments, the bowel preparation solution comprises from about 100 mL to about 1000 mL of an aqueous hypertonic solutions comprising an effective amount of sodium sulfate, an effective amount of magnesium sulfate, and an effective amount of potassium sulfate, wherein the composition does not produce any clinically electrolyte shifts in the human following consumption by the human. In some embodiments, the bowel preparation solution consists essentially of from about 100 mL to about 1000 mL of an aqueous hypertonic solutions comprising an effective amount of sodium sulfate, an effective amount of magnesium sulfate, and an effective amount of potassium sulfate, wherein the composition does not produce any clinically electrolyte shifts in the human following consumption by the human.

In some embodiments, are provided any of the methods disclosed herein, wherein the bowel cleansing solution may be administered to the human in one or more doses, or two or more doses, or three or more doses, or four or more doses, or five or more doses, or 6 or more doses, or 7 or more doses, or 8 more doses, of 9 or more doses, or 10 or more doses, or 11 or more doses, or 12 or more doses, or 13 or more doses, or 14 or more doses, or 15 or more doses, or 16 or more doses, or 17 or more doses, or 18 or more doses, or 19 or more doses, or 20 or more doses.

In some embodiments are provided any of the methods disclosed herein, wherein the human consumes at least one liter, or at least two liters, or at least three liters, or at least 4 liters of bowel cleansing solution prior to the administration of the first dose of the solid composition. In some embodiments are provided any of the methods disclosed herein, wherein the human consumes at least one liter of bowel cleansing solution prior to the administration of the first dose of the solid composition. In some embodiments are provided any of the methods disclosed herein, wherein the human consumes at least one liter, or at least two liters, or at least three liters, or at least 4 liters of bowel cleansing solution prior to the administration of the first dose of the solid composition, wherein the human is administered 8 tablets of the solid composition at least 8 hours prior to the endoscopic procedure, and wherein the human consumes the entire volume of bowel cleansing solution at least 8 hours prior to the endoscopic procedure. In some embodiments are provided any of the methods disclosed herein, wherein the human consumes at least one liter, or at least two liters, or at least three liters, or at least 4 liters of bowel cleansing solution prior to the administration of the first dose of the solid composition, wherein the human is administered 8 tablets of the solid composition at least 8 hours prior to the endoscopic procedure, and wherein the human consumes the entire volume of bowel cleansing solution at least 2 hours prior to the endoscopic procedure.

In some embodiments are provided any of the disclosed methods wherein the human is administered 8 tablets of a solid composition and consumes a total volume of 4 liters of a bowel cleansing solution according to the schedule in the table below.

| Time from | Volume of bowel | |
|---|---|---|
| consumption of | cleansing | Number of tablets of solid |
| first volume of | solution (mL) to | composition comprising 25 mg |
| bowel cleansing | be consumed | of methylene blue to be |
| solution (minutes) | by the human | administered to the human |
| 0 | 250 | 0 |
| 15 | 250 | 0 |
| 30 | 250 | 0 |
| 45 | 250 | 0 |
| 60 | 250 | 0 |
| 75 | 250 | 0 |
| 90 | 250 | 0 |
| 105 | 250 | 0 |
| 120 | 250 | 3 |
| 135 | 250 | 0 |
| 150 | 250 | 0 |
| 165 | 250 | 0 |
| 180 | 250 | 3 |
| 195 | 250 | 0 |
| 210 | 250 | 0 |
| 225 | 250 | 0 |
| 240 | Consume water | 2 |

In some embodiments, are provided any of the methods disclosed herein wherein the human is orally administered a total of 8 tablets of a solid composition in a single oral administration during the intake of a bowel cleansing preparation. In some embodiments, the 8 tablets are administered after the intake of at least one liter of the bowel cleansing preparation. In some embodiments, the 8 tablets are administered at least 8 hours prior to the endoscopic procedure. In some embodiments, the 8 tablets are administered the evening before the endoscopic procedure.

In some embodiments, are provided any of the methods disclosed herein wherein the human is orally administered a total of 8 tablets of a solid composition according to a fractionated dose regimen during the intake of a bowel cleansing preparation. In some embodiments, the fractionated dose regimen comprises two oral administrations. In some embodiments, the fractionated dose regimen comprises three oral administrations. In some embodiments, the fractionated dose regimen comprises four oral administrations s. In some embodiments, the fractionated dose regimen comprises five oral administrations. In some embodiments, the fractionated dose regimen comprises six oral administrations. In some embodiments, the fractionated dose regimen comprises seven oral administrations. In some embodiments, the fractionated dose regimen comprises eight oral administrations. In some embodiments, each oral administration comprises one to seven tablets. In some embodiments, each oral administration comprises one, or two, or three, or four, or five, or six, or seven tablets. In some embodiments, the first dose of the solid composition is administered after at least one liter of the bowel cleansing preparation. In some embodiments, the first dose of the solid composition is administered after at least two liters of the bowel cleansing preparation. In some embodiments, the first dose of the solid composition is administered after at least three liters of the bowel cleansing preparation. In some embodiments, the first dose of the solid composition is administered the whole volume of the bowel preparation has been consumed. In some embodiments, the fractionated dose regimen comprises a timeframe of about 30 minutes from an oral administration of the solid composition and the following one. In some embodiments, the fractionated dose regimen comprises a timeframe of about 60 minutes from an oral administration of the solid composition and the following one. In some embodiments, the fractionated dose regimen comprises a timeframe of about 90 minutes from an oral administration of the solid composition and the following one. In some embodiments, the fractionated dose regimen comprises a timeframe of about 120 minutes from an oral administration of the solid composition and the following one. In one embodiment is provided a method for improving the detection of pathologies in the colon of a human, comprising orally administering to the human 8 tablets of a solid composition and a volume of a bowel cleaning solution, wherein the solid composition is administered orally in three doses during the intake of the bowel cleansing solution according to the following schedule: (a) a first dose comprising administration of 3 tablets of the solid composition to the human following consumption of at least one liter of bowel cleansing solution; (b) a second dose comprising administration of 3 tablets of the solid composition to the human about 1 hour following administration the first dose of the solid composition; and (c) a third dose comprising administration of 2 tablets of the solid composition to the human about 1 hour following administration of the second dose of the solid composition.

In one embodiment, the adenoma detection rate is at least about 40%, or at least about 45%, or at least about 50%, or at least about 55%. In another embodiment, the adenoma detection rate is of about 56.29%. Such an improved adenoma detection rate (ADR) is relevant in order to significantly prevent colon rectal cancer (CRC). CRC is one of the most important causes of death for cancer worldwide (the third cause in the US, specifically), therefore, it is clear the advantages related to an improved ADR for preventing the occurrence of CRC.

In some embodiments of the present invention, the method is characterized in a detection rate of the proportion of subjects with non-polypoid lesion instead of the adenoma detection rate. In such embodiments, the detection rate of the proportion of subjects with non-polypoid lesion is at least about 30%, or at least about 35%, or at least about 40%. In another embodiment, the detection rate of the proportion of subjects with non-polypoid lesion is about 43.92%.

In some embodiments of the present invention, the method is characterized in a detection rate of the proportion of subjects with diminutive adenoma instead of the adenoma detection rate. In such embodiments, the detection rate of the proportion of subjects with diminutive adenoma is at least about 25%, or at least about 30%, or at least about 35%. In another embodiment, the detection rate of the proportion of subjects with diminutive adenoma is about 37.11%.

In another aspect, the present invention provides a method for improving the detection of pathologies in the colon, comprising orally administering to a human 4 liters of a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution;
c) 2 unit dosages of the solid composition after the intake of a 4^{th} liter of bowel cleaning solution;
   wherein each unit dosage of the solid composition contains 25 mg of methylene blue, whereby the false positive rate is not more than about 35%.

In another aspect, the present invention provides a method for improving the detection of pathologies in the colon, comprising orally administering to a human 4 liters of a bowel cleansing solution and 8 unit dosages of a solid composition, wherein the bowel cleansing solution and the 8 unit dosages of the solid composition are administered to the human according to the schedule comprising:
d) 3 unit dosages of the solid composition after the intake of 2 liters of bowel cleaning solution, preferably in about two hours;
e) 3 unit dosages of the solid composition after the intake of a 3^{rd} liter of bowel cleaning solution, preferably 1 hour after the first oral administration of the solid composition;
f) 2 unit dosages of the solid composition after the intake of a 4^{th} liter of bowel cleaning solution, preferably 1 hour after the second oral administration of the solid composition.

In one embodiment is provided a method for improving the detection of pathologies in the colon of a human, comprising orally administering to the human 8 tablets of a solid composition and a volume of a bowel cleaning solution, wherein the solid composition is administered orally in three doses during the intake of the bowel cleansing solution according to the following schedule: (a) consumption of at least one liter of bowel cleansing solution, (b) a first dose comprising administration of 3 tablets of the solid composition and a second liter of bowel cleansing solution to the human one hour following consumption of the first liter of bowel cleansing solution; (c) a second dose comprising administration of 3 tablets of the solid composition and a third liter of bowel cleansing solution to the human about 1 hour following administration the first dose of the solid composition; and (c) a third dose comprising administration of 2 tablets of the solid composition and a fourth liter of bowel cleansing solution to the human about 1 hour following administration of the second dose of the solid composition.

In one embodiment is provided a method for improving the detection of pathologies in the colon, comprising orally administering to a human 4 liters of a bowel cleansing solution and 8 dosage units of a solid composition, wherein the bowel cleansing solution and the 8 dosage units of the solid composition are administered to the human according to the schedule comprising:
a) 3 dosage units of the solid composition after the intake of at least one liter of bowel cleaning solution;
b) 3 dosage units of the solid composition 1 hour after the first oral administration of the solid composition;
c) 2 dosage units of the solid composition 1 hour after the second oral administration of the solid composition;
   wherein each oral administration of the composition is accompanied by bowel cleansing preparation or water, wherein each unit dosage of the solid composition contains 25 mg of methylene blue.

In one embodiment, the false positive rate is not more than about 30%, or not more than about 25%. In another embodiment, the false positive rate is about 22.74%.

In another aspect, the present invention provides a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liters of bowel cleaning solution;
c) 2 unit dosages of the solid composition after the intake of a 4^{th} liter of bowel cleaning solution;
   wherein each unit dosage of the solid composition contains 25 mg of methylene blue, whereby the false positive rate is not more than about 35% and the adenoma detection rate is at least about 40%.

In a further aspect, the present invention, also provides a method of flagging mucosal lesions in the colon, by orally administering at least one tablet containing methylene blue as described herein to a subject undergoing colonoscopy and in at least a single dose, a multiple dose or in a dosage regimen that is described herein. In some embodiments, the method further comprises orally administering to a human a bowel cleansing solution. Such flagging of the mucosal lesions is due to a differential uptake of the dye by the abnormal cells of the colonic mucosa, with respect to the normal ones. In one embodiment, the flagging highlights the lesions by a coloration having an intensity that is higher than the surrounding mucosa. In another embodiment, the flagging highlights the lesions by a coloration with an intensity that is lower than the surrounding mucosa. In some embodiments, the coloration is blue. In a further embodiment, the flagging allows the lesion to be stained while the surrounding mucosa remains uncolored. In another embodiment, the flagging allows the lesion to be stained on the margins only.

Such differential coloration, due to the peculiar formulation of the tablets described herein, allows a clear perception of the zones where the lesions are located in the colonic mucosa, leading to an easier visualization of the same. The ability of the methods of the present invention to flag mucosal lesions is surprising because it was a common understanding that it was not possible to obtain flagging of the mucosal lesions with respect to the surrounding healthy mucosa.

Although the dye used in certain embodiments is methylene blue, the color that is seen may be different from a visual point of view. Thus, the coloration may be a blue coloration, it does not necessarily have to be blue. The color is evidenced in different part of the lesions or of the healthy mucosa, flagging the cells; in case of lesions: margins, tops, whole lesion, peduncle depending on the type of the cells.

The present invention is suitable for detecting pathological lesions, such as precancerous, cancerous forms, interval cancers, adenomas, carcinomas, serrated lesions, dysplasias, polyps, pseudopolyps, pre-polyps, hyperplatic lesions, and the like. See also WO2014/060199.

### BRIEF DESCRIPTION OF THE FIGURES

The patent or application file contains at least one drawing executed in color. Copies of this patent or patent application publication with color drawing(s) will be provided by the Office upon request and payment of the necessary fee. As the color drawings are being filed electronically via EFS-Web, only one set of the drawings is submitted.

FIG. 1 shows the contrast enhancing efficacy of the dye according to Example 5 in perceiving the deep mucosal tissue structure, with the foci of the glands well defined and darkened in a pre-polyp alteration of the colonic mucosa.

FIG. 2 shows the semi-continuous blue line defines exactly the borders of the colonic flat lesion that the endoscopist has to take out, allowing a better resolution of the lesion intervention and extraction according to Example 5. The tissue definition is absolutely enhanced owing to the orally administered dye as disclosed herein. With the conventional spraying techniques, the same performance cannot be obtained since little time is available between spray and observation (seconds or a couple of minutes).

FIG. 3 shows a picture of a colonic lesion collected during the clinical study in Example 7. It is evident that the dye has been taken-up by the normal mucosal cells. The dye precisely highlights the features of the colonic surface, evidencing the lines and the crypts with a blue coloration. The lesion is flagged without color. The normal features of the colonic mucosa show an interruption in the zone where the lesion is located.

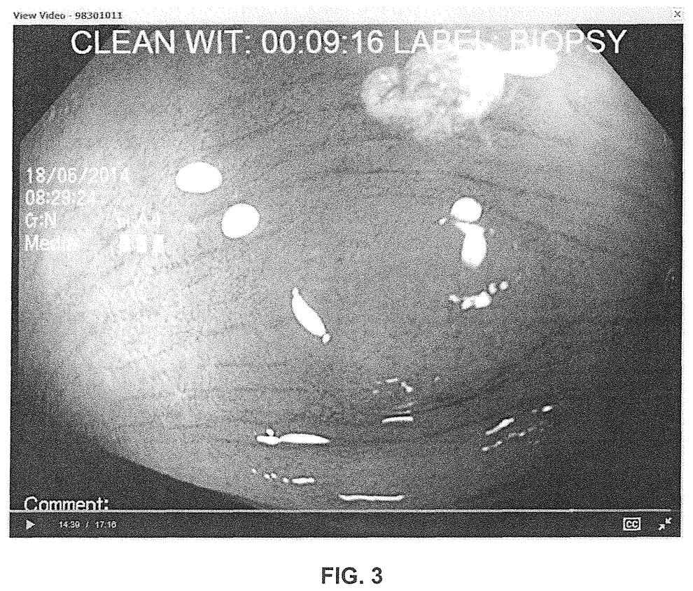

FIG. 4 shows a picture of a colonic lesion collected during the clinical study in Example 7. It is evident that the dye has been taken-up by both the pathologic and normal mucosal cells. It should be noted that the mucosal lesion has been flagged since its coloration is more intense than the surrounding healthy mucosa. Although the color is present in both the lesion and the healthy mucosa, it is clear where the lesion is (flagged with blue margins).

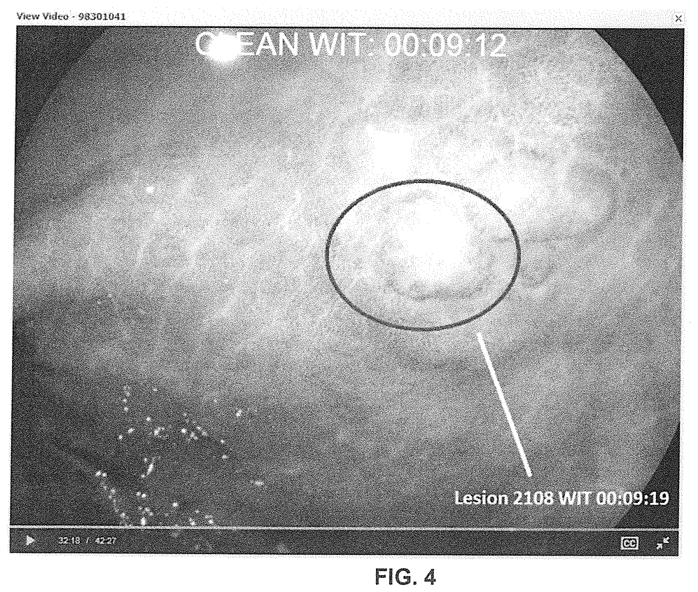

FIG. 5 shows a picture of a colonic lesion collected during the clinical study in Example 7. It is evident that the dye has been taken-up by the pathological cells of the colonic lesion. The lesion is flagged in blue color and is highlighted from the surrounding, healthy mucosa which remains uncolored. The dye precisely highlights the irregular margins of the lesion.

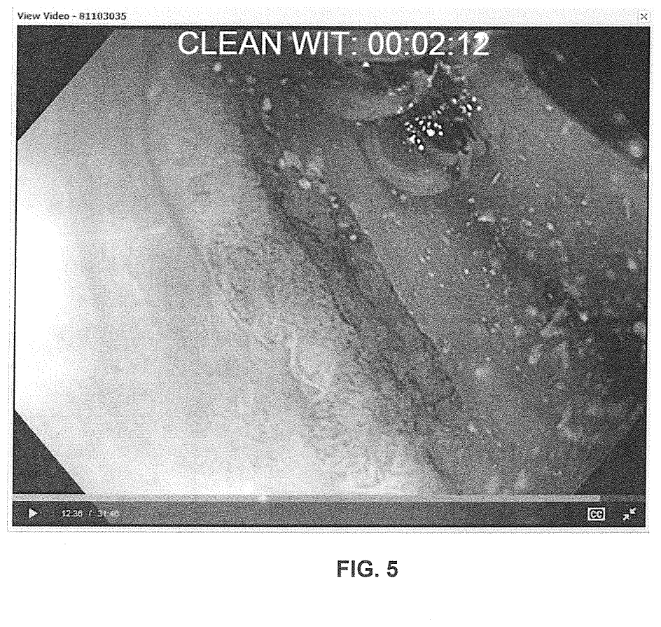

FIG. 6 shows a picture of a colonic lesion collected during the clinical study in Example 7. It is evident that the dye has been taken-up by the pathological cells of the colonic lesion while the surrounding mucosa remains uncolored. This flags the lesion and allows an immediate perception of the same. After histopathological assessment, the lesion was identified as a sessile serrated adenoma (SSA), one of the lesions of the colon more difficult to detect, and also one of the precursors of colorectal cancer (CRC).

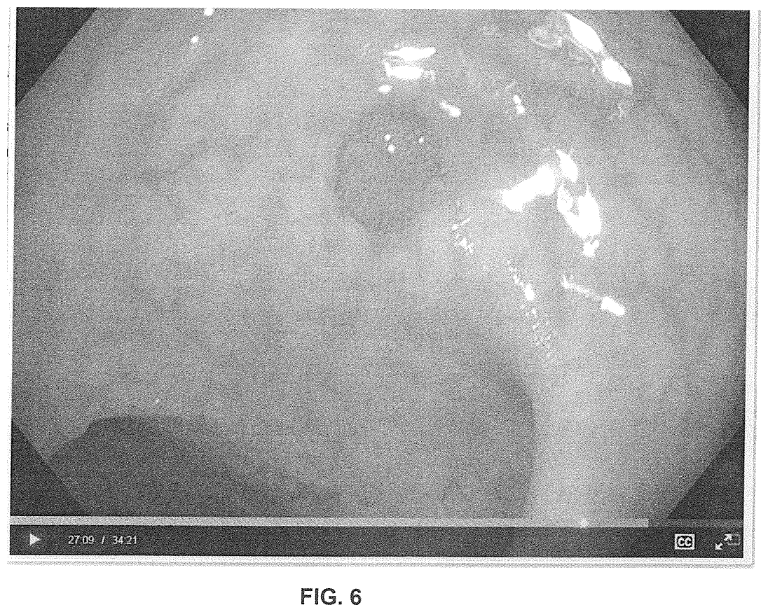

FIG. 7 shows a picture of a colonic lesion collected during the clinical study in Example 7. It is evident that the dye has been taken-up by both the pathological cells of the colonic lesion and the normal cells of the surrounding mucosa. The dye precisely highlights the features of the colonic surface, evidencing the lines and the crypts with a blue coloration. The lesion, on the contrary, is flagged with a blue color more intense than the surrounding tissues. The dye absorbed by the lesion evidences the dysplastic and disorganized structure, thereby flagging the lesion with respect to the surrounding tissues.

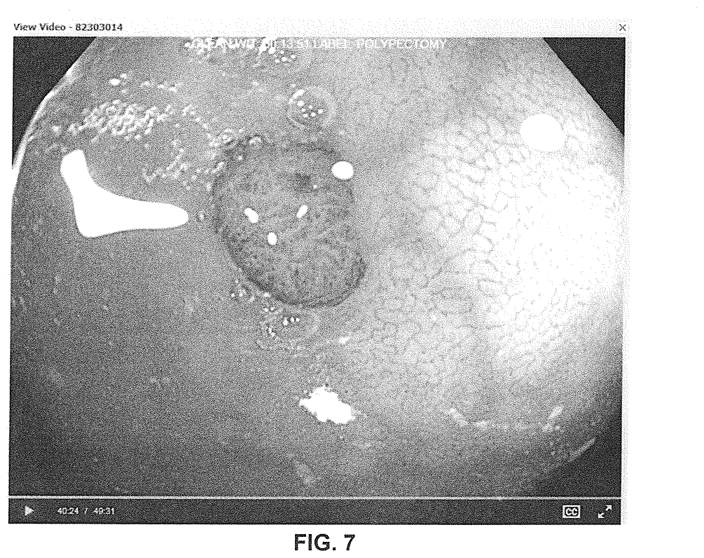

FIG. 8 shows a picture of a colonic lesion collected during the clinical study in Example 7. It is evident that the dye has been taken-up in the margins of the lesion only. The body of the lesion is uncolored, as well as the surrounding healthy mucosa. The color is concentrated along the margins, flagging where the lesion is.

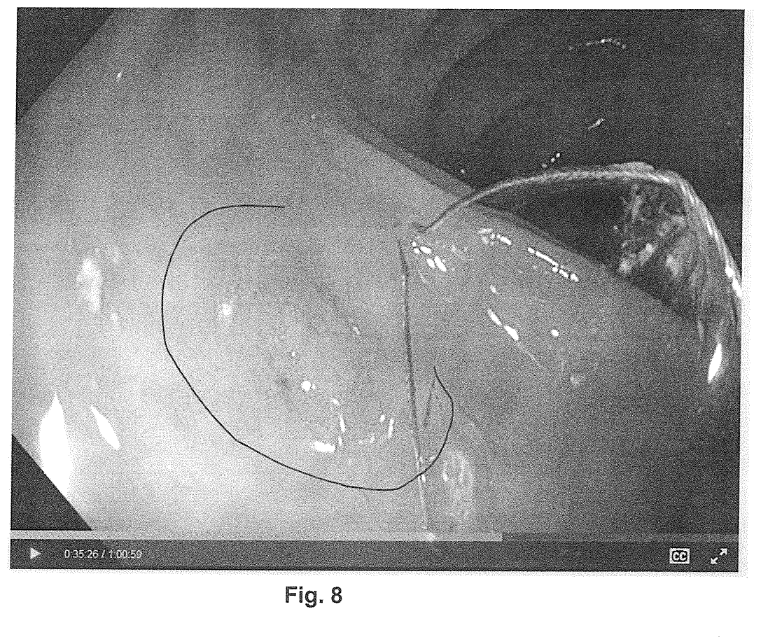

### DETAILED DESCRIPTION OF THE INVENTION

In one aspect, the present invention provides a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution;
c) 2 unit dosages of the solid composition after the intake of a 4^{th} liter of bowel cleaning solution;
   wherein each unit dosage of the solid composition contains 25 mg of methylene blue, whereby the adenoma detection rate is at least about 40%.

In one aspect, the present invention provides a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
d) 3 unit dosages of the solid composition after the intake of 2 liters of bowel cleaning solution, preferably in about two hours;
e) 3 unit dosages of the solid composition after the intake of a 3^{rd} liter of bowel cleaning solution, preferably 1 hour after the first oral administration of the solid composition;
f) 2 unit dosages of the solid composition after the intake of a 4^{th} liter of bowel cleaning solution, preferably 1 hour after the second oral administration of the solid composition.

In another aspect are provided a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of at least one liter of bowel cleaning solution;
b) 3 unit dosages of the solid composition 1 hour after the first oral administration of the solid composition;
c) 2 unit dosages of the solid composition 1 hour after the second oral administration of the solid composition;
   wherein each oral administration of the composition is accompanied by bowel cleansing preparation or water, wherein each unit dosage of the solid composition contains 25 mg of methylene blue, whereby the adenoma detection rate is at least about 40%.

In one embodiment, the adenoma detection rate is at least about 40%, or at least about 45%, or at least about 50%, or at least about 55%. In another embodiment, the adenoma detection rate is about 56.29%. Such an improved adenoma detection rate (ADR) is relevant in order to significantly prevent colon rectal cancer (CRC). CRC is one of the most important cause of death for cancer worldwide (the third cause in the US, specifically), therefore, it is clear the advantages related to an improved ADR for preventing the occurrence of CRC.

In some embodiments of the present invention, the method is characterized in a detection rate of the proportion of subjects with non-polypoid lesion instead of the adenoma detection rate. In such embodiments, the detection rate of the proportion of subjects with non-polypoid lesion is at least about 30%, or at least about 35%, or at least about 40%. In another embodiment, the detection rate of the proportion of subjects with non-polypoid lesion is about 43.92%.

In some embodiments of the present invention, the method is characterized in a detection rate of the proportion of subjects with diminutive adenoma instead of the adenoma detection rate. In such embodiments, the detection rate of the proportion of subjects with diminutive adenoma is at least about 25%, or at least about 30%, or at least about 35%. In another embodiment, the detection rate of the proportion of subjects with diminutive adenoma is about 37.11%.

In another aspect, the present invention provides a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution;
c) 2 unit dosages of the solid composition after the intake of a 4^{th} liter of bowel cleaning solution;
   wherein each unit dosage of the solid composition contains 25 mg of methylene blue, whereby the false positive rate is not more than about 35%.

In another embodiment is provided a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of at least one liter of bowel cleaning solution;
b) 3 unit dosages of the solid composition 1 hour after the first oral administration of the solid composition;
c) 2 unit dosages of the solid composition 1 hour after the second oral administration of the solid composition;
   wherein each oral administration of the composition is accompanied by bowel cleansing preparation or water, wherein each unit dosage of the solid composition contains 25 mg of methylene blue, whereby the false positive rate is not more than about 35%.

In one embodiment, the false positive rate is not more than about 30%, or not more than about 25%. In another embodiment, the false positive rate is of about 22.74%.

In another aspect, the present invention provides a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution;
c) 2 unit dosages of the solid composition after the intake of a 4^{th} liter of bowel cleaning solution;
   wherein each unit dosage of the solid composition contains 25 mg of methylene blue, whereby the false positive rate is not more than about 35% and the adenoma detection rate is at least about 40%.

In another aspect is provide a method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of at least one liter of bowel cleaning solution;
b) 3 unit dosages of the solid composition 1 hour after the first oral administration of the solid composition;
c) 2 unit dosages of the solid composition 1 hour after the second oral administration of the solid composition;
   wherein each oral administration of the composition is accompanied by bowel cleansing preparation or water, wherein each unit dosage of the solid composition contains 25 mg of methylene blue, whereby the false positive rate is not more than about 35% and the adenoma detection rate is at least about 40%.

A solid composition useful in the present invention comprises at least one dye in association with at least one physiologically acceptable excipient which comprises:
a) a matrix which comprises at least one lipophilic compound, preferably a lipophilic compound with a melting point below 90° C., and optionally at least one amphiphilic compound, in which matrix at least one dye is at least partly incorporated,
b) a matrix which comprises at least one hydrophilic compound, in which the lipophilic matrix, and optionally the amphiphilic matrix are dispersed;
c) optionally other physiologically acceptable excipients;
d) optionally a gastro-resistant coating
   for use in endoscopic diagnosis characterised in that two or more unit dosages of the solid composition are orally administered to a human according to a fractionated schedule in which a total amount from 100 to 400 mg of said at least one dye is administered to a human in the 48 hours prior to endoscopic diagnosis. For example, said at least one dye is administered to a human in the 24 hours prior to endoscopic diagnosis.

In the alternative, the matrix consists of at least one lipophilic compound, preferably a lipophilic compound with a melting point below 90° C., and optionally at least one amphiphilic compound, in which matrix at least one dye is at least partly incorporated, and the matrix consists of at least one hydrophilic compound, in which the lipophilic matrix, and optionally the amphiphilic matrix are dispersed.

Said two or more unit dosages are, for example, four, six or eight unit dosages administered in the 48 hours prior to endoscopy, such as in the 24 hours prior to endoscopy.

Useful dyes according to the present disclosure can be, for example, selected from among congo red, carmine indigo, methylene blue, toluidine blue or mixtures thereof. In some embodiments, the dye is methylene blue.

According to the disclosure herein, methylene blue can be in anhydrous or hydrated forms, such as the trihydrate form.

However, according to the disclosure other biocompatible dye substances can also be used, as long as they are provided with a toxicity profile that does not represent an obstacle to oral systemic administration thereof.

A “fractionated schedule” according to the disclosure means that the total amount of the dye to be orally administered before colonoscopy is divided in two or more unit dosages to obtain a pre-defined administration schedule. The dose fractionation can reduce the possibility that staining will be lost due to unwanted strange intestinal motility. And the dose fractionation can facilitate the spreading of the blue staining matrices.

The endoscopic diagnosis as disclosed herein is directed to the gastro-intestinal tract, such as the colon (colon endoscopy or colonoscopy). According to the anatomical classification, the colon is divided into four (4) regions of interest (ROI), namely (1) ascending colon (AC), (2) transverse colon (TC), (3) descending colon (DC), and (4) rectosigmoid (RES).

As disclosed herein, the total dose amount of said at least one dye is, for example, from 50 to 500 mg, such as from 100 to 400 mg, such as from 100 to 250 mg, and further such as 200 mg.

As disclosed herein, the unit dosage of the composition contains, for example, from 20 to 200 mg by weight of the at least one dye. For example, said unit dosage contains about 25 mg or about 50 mg, such as 25 mg or 50 mg, by weight of said at least one dye.

According to an embodiment disclosed herein, eight unit dosages of the composition, each containing about 25 mg, such as 25 mg, by weight of said at least one dye, are administered to said human in the 48 hour period prior to endoscopic diagnosis.

According to another embodiment disclosed herein, six unit dosages of the composition, each containing about 25 mg, such as 25 mg, by weight of said at least one dye, are administered to said human in the 48 hour period prior to endoscopic diagnosis.

According to a yet one other embodiment disclosed herein, four unit dosages of the composition of the invention, each containing about 25 mg, such as 25 mg, by weight of said at least one dye, are administered to said human in the 48 hour period prior to endoscopic diagnosis.

According to a further embodiment disclosed herein, four unit dosages of the composition, each containing about 50 mg, such as 50 mg, by weight of said at least one dye, are administered to said human in the 48 hour period prior to endoscopic diagnosis.

According to a yet further embodiment disclosed herein, two unit dosages of the composition disclosed herein, each containing about 200 mg, such as 200 mg, by weight of said at least one dye, are administered to said human in the 48 hour period prior to endoscopic diagnosis.

In some embodiments disclosed herein, the tablets comprising the solid composition are to be orally administered to the human, wherein the human swallows the tablets whole, without crushing, breaking or chewing the tablets.

In some embodiments, the administration of the tablets comprising the solid composition are contraindicated for administration to humans that a have a hypersensitivity to methylene blue or any other thiazine dye, or a severe hypersensitivity to methylene blue or any other thiazine dye, or humans having a glucose-6-phosephate dehydrogenase (G6PD) deficiency, including humans at risk of developing haemolytic anaemia. In this case laboratory testing may show Heinz bodies, elevated indirect bilirubin and low haptoglobin, but the Coombs test is negative. The anemia may require red blood cell transfusions

Anaphylactic reactions to methylene blue class products have been reported in some humans administered methylene blue. Humans treated with tables comprising the solid composition should be monitored for anaphylaxis. If anaphylaxis or other severe hypersensitivity reactions (e.g. angioedema, urticaria, bronchospasm) should occur, the use of the tablets comprising the solid composition may be discontinued. Tablets comprising the solid composition may be contraindicated in humans who have experienced anaphylaxis or other severe hypersensitivity reactions to a methylene blue class product in the past.

In some embodiments, the tablets comprising the solid composition should not be used in humans that are pregnant, breastfeeding or lactating.

In some embodiments, the tablets comprising the solid composition should be used with caution in individuals with severe renal insufficiency and/or hepatic impairment.

In some embodiments, administration of the tablets comprising the solid composition to humans may cause symptoms in the humans such as migraine, dizziness, balance disorder, somnolence, confusion and disturbances in vision. Humans administered the tablets comprising the solid composition may be advised to refrain from driving or engaging in hazardous occupations or activities such as operating heavy or potentially dangerous machinery until such adverse reactions have resolved.

Methylene blue inhibits a range of CYP isozymes in vitro, including 1A2, 2B6, 2C8, 2C9, 2C19, 2D6, and 3A4/5. Methylene blue induces CYP isozymes 1A2 and 2B6 in human hepatocytes culture, whereas it does not induce 3A4 at nominal concentrations up to 40 μM. These interactions could be more pronounced with narrow therapeutic index drugs that are metabolized by one of these enzymes (e.g., digoxin, warfarin, phenytoin, alfentanil, cyclosporine, dihydroergotamine, ergotamine, fentanyl, pimozide, quinidine, sirolimus, and tacrolimus). However, the clinical relevance of these in vitro interactions is unknown.

Based on in vitro studies, methtylene blue was found to be a possible substrate of the membrane transport proteins P-gp and OAT3 and drugs which are inhibitors of these transporters have the potential to decrease excretion efficiency of methylene blue. Caution should be taken when methylene is co-administered with agents such as cyclosporine A, ritonavir, saquinavir, amiodarone, alectinib, probenecid and novobiocin.

Based on in vitro studies methylene blue was found to likely act as a weak inhibitor of P-gp, therefore as methylene blue has the potential to increase plasma concentrations of co-administered substrates of this transporter (digoxin, topotecan, sirolimus, everolimus, nilotinib and lapatinib), appropriate monitoring is recommended.

The dissolution of the solid compositions disclosed herein may be pH dependent, and the release properties and uptake of methylene blue may be altered in human when administered following administration of gastric acid reducing agents to the human (e.g., PPIs, H2-blockers, and antacids).

In some embodiments, the total dose of the tablets comprising the solid composition may be taken orally during the intake of the bowel cleansing preparation and should be completed the evening prior to the colonoscopy to ensure there is enough time for the tablets to reach the colon and locally release the methylene blue prior to the colonoscopy.

As disclosed herein, to facilitate the mucosal observation through the endoscope by the endoscopist, said human, prior to endoscopic diagnosis, can be subjected to a bowel cleansing preparation by the administration of bowel cleansing solution to quantitatively remove the stool and mucous residuals. This cleansing operation is carried out generally in the 48 hour period prior to endoscopic diagnosis, such as in the 24 hour period prior to endoscopic diagnosis or, as found to be practical for carrying out a colonoscopy in the late afternoon, also in the same day.

The colon cleansing preparation could be administered by drinking the volume fractions of the cleansing solution consecutively during the day before or, with the so-called “split” version, by dividing the administration of the cleansing solution volume in two parts, one to be administered the day before the colonoscopy and one to be administered in the morning of the day in which the colonoscopy is to be subsequently performed.

The bowel cleansing solution is used for cleaning and washing the intestinal tract and mucosa before the endoscopic diagnosis. The bowel cleansing solution is, for example. a saline and/or polyethylenglycol (PEG) aqueous solution, such as a polyethylene glycol aqueous solution. As a further example, said aqueous solution contains, excluding water, from 50% to 95% by weight of polyethylene glycol, sometimes also including in that solution, salts and flavours, such as sodium salts, potassium salts, ascorbic acid, and mixtures thereof. For example, sodium sulphate, sodium sulphate anhydrous, sodium chloride, sodium ascorbate, sodium bicarbonate, sodium salt of ascorbic acid, potassium sulphate, potassium chloride and mixtures thereof can be used. As a further example, the bowel cleansing solution is an aqueous solution of commercially available products sold under such names as Moviprep® or Golytely®, Nulytely®, or Halflytely®, or Movicol®, or Macro-P®, or Colirei®, or Isocolan® or Selg 1000®.

However, as disclosed herein, also other bowel cleansing solutions or preparations can be used, as long as they are provided with a toxicity profile that does not represent an obstacle to oral systemic administration thereof. For example, bowel cleansing solution containing only salts or other small chemical laxatives, but not PEG, are available on the market under the brands Phospho-Lax® or Picoprep® or Suprep®. Also different bowel preparation procedures can be used.

As disclosed herein, the cleansing solution can be administered in a total amount of four litres, which can be fractionated in one or more unit dosages, for example, in four unit dosages of about one litre each.

The solid composition, as disclosed herein, can be thus administered together and/or after the intake of each unit dosage of said bowel cleansing solution, prior to the endoscopic diagnosis. Afterwards, still water can also be additionally administered, if necessary.

As disclosed herein, four unit dosages of the composition, each containing about 25 mg, such as 25 mg, by weight of said at least one dye, are orally administered to a human according to a fractionated schedule in which a total amount of about 100 mg, such as 100 mg, of said at least one dye is administered to said human in the 48 hour period prior to the endoscopic diagnosis in:
1 solid oral composition after intake of the 1^{st} litre of bowel cleansing solution;
1 solid oral composition after intake of the 2^{nd} litre of bowel cleansing solution;
1 solid oral composition after intake of the 3^{nd} litre of bowel cleansing solution; and
1 solid oral composition after intake of the 4^{nd} (and last) litre of bowel cleansing solution.

As disclosed herein, eight unit dosages of the composition, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
2 solid oral compositions after intake of the 1^{st} litre of bowel cleansing solution;
2 solid oral compositions after intake of the 2^{nd} litre of bowel cleansing solution;
2 solid oral compositions after intake of the 3^{nd} litre of bowel cleansing solution; and
2 solid oral compositions after intake of the 4^{nd} (and last) litre of bowel cleansing solution.

For example, eight unit dosages of the composition as disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
2 solid oral compositions after intake of the 2^{nd} litre of bowel cleansing solution
3 solid oral compositions after intake of the 3^{nd} litre of bowel cleansing solution; and
3 solid oral compositions after intake of the 4^{nd} (and last) litre of bowel cleansing solution.

As a further example, eight unit dosages of the composition disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
4 solid oral compositions after intake of the 2^{nd} litre of bowel cleansing solution;
4 solid oral compositions after intake of the 3^{nd} litre of bowel cleansing solution; and
0 solid oral compositions after intake of the 4^{th} litre of bowel cleansing solution.

As a yet further example, eight unit dosages of the composition as disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
3 solid oral compositions after intake of the 2^{nd} litre of bowel cleansing solution;
3 solid oral compositions after intake of the 3^{nd} litre of bowel cleansing solution; and
2 solid oral compositions after intake of the 4^{th} litre of bowel cleansing solution.

As further disclosed within, four unit dosages of the composition as disclosed herein, each containing about 25 mg, such as 25 mg, by weight of said at least one dye, are orally administered to a human according to a fractionated schedule in which a total amount of about 100 mg, such as 100 mg, of said at least one dye is administered to said human in the 48 hour period prior to the endoscopic diagnosis in:
0 solid oral composition after intake of the 1^{st} litre of bowel cleansing solution;
1 solid oral composition after intake of the 2^{nd} litre of bowel cleansing solution;
1 solid oral compositions after intake of the 3^{nd} litre of bowel cleansing solution; and
2 solid oral compositions after intake of the 4^{nd} (and last) litre of bowel cleansing solution.

As further disclosed within, two unit dosages of the composition as disclosed herein, each containing about 200 mg, such as 200 mg, by weight of said at least one dye, are orally administered to a human according to a fractionated schedule in which a total amount of about 400 mg, such as 400 mg, of said at least one dye is administered to said human in the 48 hour period prior to the endoscopic diagnosis in:
0 solid oral composition after intake of the 1^{st} litre of bowel cleansing solution;
1 solid oral composition after intake of the 2^{nd} litre of bowel cleansing solution;
1 solid oral compositions after intake of the 3^{nd} litre of bowel cleansing solution; and
0 solid oral compositions after intake of the 4^{nd} (and last) litre of bowel cleansing solution.

As disclosed herein, four unit dosages of the composition, each containing about 25 mg, such as 25 mg, by weight of said at least one dye, are orally administered to a human according to a fractionated schedule in which a total amount of about 100 mg, such as 100 mg, of said at least one dye is administered to said human in the 48 hour period prior to the endoscopic diagnosis in:
1 solid oral composition after intake of the 1^{st} litre of bowel cleansing solution;
1 solid oral composition after intake of the 2^{nd} litre of bowel cleansing solution, preferably 1 hour after the first oral administration of the solid composition;
1 solid oral composition after intake of the 3^{nd} litre of bowel cleansing solution, preferably 1 hour after the second oral administration of the solid composition; and
1 solid oral composition after intake of the 4^{nd} (and last) litre of bowel cleansing solution, preferably 1 hour after the third oral administration of the solid composition.

As disclosed herein, eight unit dosages of the composition, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
2 solid oral compositions after intake of the 1^{st} litre of bowel cleansing solution;
2 solid oral compositions after intake of the 2^{nd} litre of bowel cleansing solution, preferably 1 hour after the first oral administration of the solid composition;
2 solid oral compositions after intake of the 3^{nd} litre of bowel cleansing solution, preferably 1 hour after the second oral administration of the solid composition; and
2 solid oral compositions after intake of the 4^{nd} (and last) litre of bowel cleansing solution, preferably 1 hour after the third oral administration of the solid composition.

For example, eight unit dosages of the composition as disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
2 solid oral compositions after intake of the 2^{nd} litre of bowel cleansing solution, preferably 1 hour after the first oral administration of the bowel cleansing solution;
3 solid oral compositions after intake of the 3^{nd} litre of bowel cleansing solution, preferably 1 hour after the first oral administration of the solid composition; and
3 solid oral compositions after intake of the 4^{nd} (and last) litre of bowel cleansing solution, preferably 1 hour after the second oral administration of the solid composition.

As a further example, eight unit dosages of the composition disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
4 solid oral compositions after intake of the 2^{nd} litre of bowel cleansing solution, preferably 1 hour after the first oral administration of the bowel cleansing solution;
4 solid oral compositions after intake of the 3^{nd} litre of bowel cleansing solution, preferably 1 hour after the first oral administration of the solid composition; and
0 solid oral compositions after intake of the 4^{th} litre of bowel cleansing solution, preferably 1 hour after the second oral administration of the solid composition.

As a yet further example, eight unit dosages of the composition as disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 48 hour period prior to the endoscopic diagnosis in:
0 solid oral compositions after intake of the 1^{st} litre of bowel cleansing solution;
3 solid oral compositions after intake of the 2^{nd} litre of bowel cleansing solution, preferably 1 hour after the first oral administration of the bowel cleansing solution;
3 solid oral compositions after intake of the 3^{nd} litre of bowel cleansing solution, preferably 1 hour after the first oral administration of the solid composition; and
2 solid oral compositions after intake of the 4^{th} litre of bowel cleansing solution, preferably 1 hour after the second oral administration of the solid composition.

As further disclosed within, four unit dosages of the composition as disclosed herein, each containing about 25 mg, such as 25 mg, by weight of said at least one dye, are orally administered to a human according to a fractionated schedule in which a total amount of about 100 mg, such as 100 mg, of said at least one dye is administered to said human in the 48 hour period prior to the endoscopic diagnosis in:
0 solid oral composition after intake of the 1^{st} litre of bowel cleansing solution;
1 solid oral composition after intake of the 2^{nd} litre of bowel cleansing solution, preferably 1 hour after the first oral administration of the bowel cleansing solution;
1 solid oral compositions after intake of the 3^{nd} litre of bowel cleansing solution, preferably 1 hour after the first oral administration of the solid composition; and
2 solid oral compositions after intake of the 4^{nd} (and last) litre of bowel cleansing solution, preferably 1 hour after the second oral administration of the solid composition.

As further disclosed within, two unit dosages of the composition as disclosed herein, each containing about 200 mg, such as 200 mg, by weight of said at least one dye, are orally administered to a human according to a fractionated schedule in which a total amount of about 400 mg, such as 400 mg, of said at least one dye is administered to said human in the 48 hour period prior to the endoscopic diagnosis in:
0 solid oral composition after intake of the 1^{st} litre of bowel cleansing solution;
1 solid oral composition after intake of the 2^{nd} litre of bowel cleansing solution, preferably 1 hour after the first oral administration of the bowel cleansing solution;
1 solid oral compositions after intake of the 3^{nd} litre of bowel cleansing solution, preferably 1 hour after the first oral administration of the solid composition; and
0 solid oral compositions after intake of the 4^{nd} (and last) litre of bowel cleansing solution, preferably 1 hour after the second oral administration of the solid composition.

As even further disclosed herein, six unit dosages of the composition, each containing about 25 mg, such as 25 mg, by weight of said at least one dye, are orally administered to a human according to a fractionated schedule in which a total amount of about 150 mg, such as 150 mg, of said at least one dye is administered to said human in the 48 hour period prior to the endoscopic diagnosis in:
2 solid oral composition at the beginning of bowel preparation, before intake of the 1^{st} litre of bowel cleansing solution;
2 solid oral compositions after intake of the 1^{st} litre of bowel cleansing solution, preferably 1 hour after the first oral administration of the solid composition;
2 solid oral compositions after intake of the 2^{nd} litre of bowel cleansing solution, preferably 1 hour after the second oral administration of the bowel cleansing solution;
0 solid oral compositions after intake of the 3^{nd} litre of bowel cleansing solution, preferably 1 hour after the second oral administration of the solid composition; and
0 solid oral compositions after intake of the 4^{nd} (and last) litre of bowel cleansing solution, preferably 1 hour after the third administration of the bowel cleansing solution.

As even further disclosed herein, six unit dosages of the composition, each containing about 25 mg, such as 25 mg, by weight of said at least one dye, are orally administered to a human according to a fractionated schedule in which a total amount of about 150 mg, such as 150 mg, of said at least one dye is administered to said human in the 48 hour period prior to the endoscopic diagnosis in:
2 solid oral composition at the beginning of bowel preparation, before intake of the 1^{st} litre of bowel cleansing solution;
2 solid oral compositions after intake of the 1^{st} litre of bowel cleansing solution;
2 solid oral compositions after intake of the 2^{nd} litre of bowel cleansing solution
0 solid oral compositions after intake of the 3^{nd} litre of bowel cleansing solution; and
0 solid oral compositions after intake of the 4^{nd} (and last) litre of bowel cleansing solution.

As yet another further example, the above indicated administration schedule can be carried out applying also the “split” bowel cleansing procedure. In such a case, the tablet administration is split over the two days of bowel cleansing preparation, maintaining the relevant schedule here described. Examples of the split preparation, according to further example disclosed herein, are here below detailed:
eight unit dosages of the composition disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 24 hour period prior to the endoscopic diagnosis in a split preparation procedure, where:
3 solid oral compositions after intake of the 1^{st} litre of bowel cleansing solution the day before colonoscopy;
3 solid oral compositions after intake of the 2^{nd} litre of bowel cleansing solution the day before colonoscopy;
2 solid oral compositions after intake of the 3^{nd} litre of bowel cleansing solution the same day of colonoscopy; and
0 solid oral compositions after intake of the 4^{th} litre of bowel cleansing solution the same day of colonoscopy.

Alternatively, as a further example, eight unit dosages of the composition disclosed herein, each containing about 25 mg, such as 25 mg, by weight of said at least one dye, are orally administered to a human according to a fractionated schedule in which a total amount of about 200 mg, such as 200 mg, of said at least one dye is administered to said human in the 24 hour period prior to the endoscopic diagnosis in a split preparation procedure, where:
0 solid oral compositions after intake of the 1^{st} litre of bowel cleansing solution the day before colonoscopy;
6 solid oral compositions during the intake of the 2^{nd} litre of bowel cleansing solution the day before colonoscopy;
2 solid oral compositions after intake of the 3^{nd} litre of bowel cleansing solution the same day of colonoscopy; and
0 solid oral compositions after intake of the 4^{th} litre of bowel cleansing solution the same day of colonoscopy.

In a further aspect, the present invention also provides a method of flagging mucosal lesions in the colon, by orally administering one or more tablets containing methylene blue as described herein in at least a single dose, a multiple dose or in a dosage regimen described herein to a subject undergoing colonoscopy. In some embodiments, the method further comprises orally administering to a human a bowel cleansing solution. Such flagging of the mucosal lesions is due to a differential uptake of the dye by the abnormal cells of the colonic mucosa, with respect to the normal ones. In one embodiment, the flagging highlights the lesions by a coloration having an intensity that is higher than the surrounding mucosa. In another embodiment, the flagging highlights the lesions by a coloration with an intensity that is lower than the surrounding mucosa. In some embodiments, the coloration is blue. In a further embodiment, the flagging allows the lesion to be stained while the surrounding mucosa remains uncolored. In another embodiment, the flagging allows the lesion to be stained on the margins only.

Such differential coloration, due to the peculiar formulation of the tablets described herein, allows a clear perception of the zones where the lesions are located in the colonic mucosa, leading to an easier visualization of the same.

Such differential coloration provided by methods of the invention is ensured by an increase of the contact time between the dye and the mucosa. Thanks to the formulation, the dye acts locally in the colon and has a sufficient time to be taken-up by the cells of the mucosa which differentiates the present invention from prior techniques, such as spraying the dye during the endoscopic examination (known in the art as “chromoendoscopy”), which does not provide a sufficient time to the dye to be absorbed by the cells. This insufficient time of contact may lead to the endoscopist missing some colonic lesions, because according to this prior art technique it is possible that the dye is absorbed to the same extent by the abnormal cells as by the normal cells. This result may occur because the time is not sufficient to allow the proper interaction between the dye and the mucosa. The ability of the methods of the present invention to flag mucosal lesions is surprising because it was a common understanding that it was not possible to obtain flagging of the mucosal lesions with respect to the surrounding healthy mucosa.

Without being bound by any theory or hypothesis, it is believed that the flagging aspect of the present invention may be due to the fact that methylene blue is a vital dye and therefore it has different absorption times depending on the different types of cells. Being vital it has the possibility to be actively absorbed by the cells, where the absorption/de-absorption time of the pathological cells could be different, for example different between pre-neoplastic and neoplastic cells.

The solid composition disclosed herein can be a controlled release composition. The expression “controlled release” of the composition disclosed herein is used to indicate a composition capable of releasing the dye in a selective site-time manner, i.e. progressive in the areas of interest. Thus, such expression comprises the “prolonged, sustained, extended, delayed or modified” release definition.

The technology suitable for the formulation of controlled release composition disclosed herein can be selected from the colonic specific release technologies, utilized with matrix structures, and the reservoir structure as systems, using dissolution controlling mechanisms and technologies known in the art, such as diffusion, swelling, and macromolecular relaxation.

The oral composition disclosed herein can be formulated according to the multimatrix technology commercially known under the trade mark MMX®, described in the international patent applications WO 2011/107945, WO 00/76481 and WO 00/76478 and U.S. Pat. No. 8,545,811, the disclosures of which relevant to multimatrix technology are specifically incorporated by reference herein.

Suitable lipophilic compounds as disclosed herein can be selected from saturated, unsaturated and hydrogenated long chain alcohols, saturated and unsaturated and hydrogenated fatty acids, salts thereof, esters and amides, mono-, di- and triglycerides of fatty acids, polyethoxylated derivatives thereof, waxes, ceramides, cholesterol, cholesterol derivatives and mixtures thereof having a melting point lower than 90° C., such as from 40 to 90° C., and further such as from 60 to 70° C.

Suitable amphiphilic compounds as disclosed herein can be selected from among polar lipids of type I and II (lecithin, phosphatidylcholine, phosphatidylethanolamine, and mixtures thereof), ceramides, glycol alkyl ethers (such as for example, diethylene glycol monomethyl ether), alkyl sulfate and sulfosuccinate salts, and mixtures thereof.

Suitable hydrophilic compounds as disclosed herein can be chosen from compounds forming a hydrogel (i.e., compounds which form a hydrogel on contact with aqueous solvents), such as those selected from among polymers and copolymers of acrylic acid, copolymers of methacrylic acid, alkyl vinylpolymers, alkyl celluloses, hydroxyalkyl celluloses, carboxyalkyl cellulose, modified and/or plurisubstituted celluloses, polysaccharides, dextrins, pectins, starches, complex starches and starch derivatives, alginic acid, synthetic rubber, natural rubber, polyalcohols and mixtures thereof.

Hydrogels are compounds which when passing from the dry state to the hydrated one undergo so-called “molecular relaxation”, namely a remarkable increase in mass and weight following the coordination of a large number of water molecules by the polar end groups present in the polymeric chains of the excipients themselves.

A suitable gastro-resistant coating, as disclosed herein, can be chosen from polymers of acrylic acid, polymers of methacrylic acid, copolymers of acrylic acid, copolymers of methacrylic acid, cellulose derivatives (such as for example cellulose acetate phthalate) hydroxybutyrate-based polymers, shellac and mixtures thereof. Such gastro-resistant coatings of the invention can also be combined with plasticisers, opacifiers, dyes and mixtures thereof.

The administration of a controlled release composition as disclosed herein actually allows releasing the dye contained in the composition precisely starting from the gastrointestinal segment intended to be subjected to endoscopic evaluation, such as in the intestinal regions and even further such as in the colonic regions.

The composition as disclosed herein is formulated in forms chosen from tablets, capsules, granules, microgranules, and pellets, such as in the form of a coated tablet, further such as in the form of gastro-protected tablets.

The capsule form disclosed herein may in turn contain granules, microgranules and/or pellets.

For example, the composition described herein may be formulated in the form of gastro-resistant tablets or in the form of a capsule containing gastro-resistant granules, gastro-resistant microgranules and/or gastro-resistant pellets.

Furthermore, the composition disclosed herein may be formulated in a double layer form, such as a double layer tablet.

As disclosed herein, in case the of colonoscopy, two or more unit dosages of the compositions disclosed herein may be provided for the oral administration of two or more unit dosages of the compositions described herein, such as a controlled release tablet, so as to prevent the dye from being dispersed into areas of the digestive tract not intended to be subjected to colonoscopy, such as, for example, the stomach, duodenum and jejunum.

For the preparation of controlled release compositions, one or more dyes can be formulated alongside substances capable of imparting progressive or massive or controlled or prolonged dissolution properties to the formulation. In addition, the formulation is coated with substances capable of dissolving solely upon reaching a specific pH, generally running from pH 5 to pH 7, that pH being typical of the section intended to be subject to the intestinal endoscopic evaluation.

Upon reaching the intestinal section of interest, characterised by a specific pH value at which the gastro-protective coating starts dissolving, the dissolution of the dye can be controlled in terms of speed so as to ensure that it occurs within the time required by the intestinal transit, such as the time to reach the colon, generally running from 4 to 24 hours.

As disclosed herein, the dye/s is/are first mixed or granulated with the material capable of forming a lipophilic matrix, such as in the presence of one or more amphiphilic substances with surfactant properties, and lastly this matrix of powders, at any degree of aggregation, is inserted into a dominant structure formed by polymers or copolymers of the hydrophilic type, also known as hydrogels, in the anhydrous state or with some residual moisture value.

Alternatively, still according to a typical application of this technology, the dye/s should be first mixed or granulated with the material capable of forming a lipophilic matrix, and after granulation this matrix structure, at any degree of aggregation, is inserted into a dominant structure formed by polymers or copolymers of hydrophilic type in anhydrous state or with some residual moisture value in the presence, for example, of one or more amphiphilic substances with surfactant properties. Subsequently the final mixture is subjected to compression.

A gastro-protective coating film, capable of preventing the dissolution of the composition in a strongly acid environment, can be lastly applied to the surface of the compositions.

Upon swallowing, such a multimatrix coated composition can be protected from contact with gastric and intestinal acids until reaching an environment with suitable pH, such as greater than 5 or 7, where the gastro-protective coating is solubilised and where the dissolution program—which will lead it to progressively distribute the dye inserted in the formulation simultaneously with the progress of transit within the digestive cavity—starts.

The endoscopic diagnosis disclosed herein is aimed at the diagnosis of inflammatory, ulcerative, pre-neoplastic, dysplastic and/or neoplastic pathologies and/or alterations of the gastrointestinal tract, such as of the colon and further such as the right part of the colon.

For example, the endoscopic diagnostic evaluation disclosed herein can be aimed at the diagnosis of cancerous forms, precancerous forms, interval cancers, adenomas, carcinomas, serrated lesions, dysplasias, polyps, pseudopolyps, pre-polyps hyperplastic lesions and different inflammatory pathologies and/or lesions of the gastrointestinal tract, such as of the colon and further such as of the right part of the colon.

The endoscopic diagnosis of the right part of the colon can also be aimed at the diagnosis of right colon adenomas, right colon polyps, serrated adenomas and right serrated lesions or interval cancers.

An interval cancer relates to lesions able to become cancers (tumours) in the time between two consecutive colon endoscopies (colonoscopies). Such time generally corresponds to a period of 2-5 years.

The oral composition disclosed herein can be aimed to increase and to improve the diagnosis of those small size lesions and flat lesions that are mostly missed during white light colonoscopy. As used herein, the term “small size” is a size equal to or less than 10 mm, such as equal or less than 5 mm. For example, polyps, adenomas and serrated lesion of the right colon of size less than 5 mm in diameter are considered to be “small size.”

The size is determined as the diameter of lesion estimated or measured by using a standard foreign body forceps.

These right colon lesions are in fact considered difficult to be seen and detected in this field, because of the anatomical conformation of the colon mucosal tissues and the possibility to have an unclean mucosal surface, that would make the lesion's detection very difficult in standard white light colonoscopy practice.

Also, the smaller colon lesions are the more difficult to be selected because of the possibility to be confused with the colonic plicas, as well as the possibility of having an unclean mucosal surface that hides such smaller lesions, thus making those smaller lesions difficult to detect.

As disclosed herein, the endoscopic diagnosis can also be aimed at the diagnosis of the above mentioned pathologies and/or lesions in a human previously suffering from at least another inflammatory pathology as, for example, Inflammatory Bowel Disease (IBD), Ulcerative Colitis or Crohn's Disease.

In that case, said human is indicated to be a “more risky patient”. In this kind of patients, in fact, the risk of subsequent pathologies and/or lesions of the intestinal and colonic mucosa is much higher than normal because the mucosa is affected by chronic flogistic processes that in the long-term may be associated with uncontrolled cell proliferation and neoplastic development. Particularly, the risk significantly increases at the colonic level where for example colon carcinoma and/or colon dysplasia and/or intraepithelial neoplasias can more likely arise in patients with long-standing ulcerative colitis and Crohn's disease.

A first advantage of the oral composition disclosed herein is to provide an improved staining quality and staining efficacy in the area to be investigated by the endoscopic diagnostic evaluation, such as the colon regions (ascending, descending, rectosigmoid and transverse colon) and even further such as the right part of the colon.

This improved staining quality is related to a number of different factors. First, the dye is quite homogenously delivered throughout the entire length of the bowel according to the multi-matrix delivery system and the specific schedule of dye administration which ensures long-lasting and anatomically consistent availability of the coloring substance. Second and foremost, the disclosure herein allows for the first time a certain interval time between the dye contact with the colonic mucosa and the endoscopic procedure. This interval time is relevant, allowing for proper dye absorption in the mucosa which becomes consistently coloured thanks to the incorporation of the blue substance into the cells. Selective dye absorption is considered the pivotal mechanism of action of vital dyes like methylene blue.

Indeed this absorption and the consequent enhanced contrast is minimally obtained when the dye is sprayed during the endoscopic procedures. The absorption is maximized when a certain interval occurs between dye delivery and endoscopic procedure.

The third factor leading to an improved staining is strictly related to colonic anatomy. Indeed the right colon has a larger lumen and a greater mucosal surface as compared to other colonic segments.

According to these facts and because of a gravity issue (during the endoscopic procedure the patients lay down in a supine position) when the dye is sprayed at the time of endoscopic procedure, the dye tends to distribute in a patchy mode, for example, in the most downward part of the mucosa (because of gravity).

Differently from this situation, in the condition of a targeted oral delivery of the dye with an MMX mechanism, at least 5 hours before the procedure, the availability of a significant dosage of the dye and the presence of abundant aqueous material (the bowel prep solution), taken together with peristaltic movements of the right colon, optimize the diffusion of the dye and contact of the dye with the different mucosa segments of the right colon.

Once the colonic mucosa is consistently and persistently coloured with methylene blue, the resulting diagnostic advantage is an increased ability to detect mucosal abnormalities according to different actions specifically related to the dye. First and foremost, areas of mucosa with inflammatory or neoplastic changes tend to decrease the uptake of the dye thus resulting in unstained areas which are easily distinguished (during the endoscopic procedures) from normal mucosa which exhibits a homogeneous staining pattern.

Another advantage of the oral composition disclosed herein is to provide an improved detection of the pathological and/or not pathological lesions in the area to be investigated by the endoscopic diagnosis, such as the colon regions in all its anatomical segments (ascending, descending, rectosigmoid and transverse colon). For example, the right part of the colon can be the more accurately stained area.

The oral composition disclosed herein allows, thanks to a different uptake of the dye in the intercellular and intracellular spaces, a contrast enhancing efficacy of the dye in perceiving the deep mucosal tissue structure with the cripta and the gland ducts, thus improving the exact definition of the lesions and/or the borders of the lesions that the endoscopist has to identify and take out. An improved definition of the mucosal tissue structure and organization of the lesions is ensured, allowing for early detection of the lesions.

The better definition of the lesions provided by the oral composition and administration schedule disclosed herein facilitates increased specificity and sensitivity of the detection of the lesions, thus reducing the occurrence of false-negatives and false-positives and allowing pathological or malignant areas to be more correctly identified and detected. In other words, the specific oral solid composition disclosed herein and the administration schedule of the solid composition defined herein provide the improved contrast of the dye on the mucosa tissue structures.

In particular, the oral solid composition and administration schedule disclosed herein enable very early detection of adenomas, colon dysplasias and colon carcinomas, particularly of those resulting from previous ulcerative colitis or Crohn's disease.

A further advantage of the oral solid composition and administration schedule disclosed herein is to provide a maximized local bioavailability of the dye and an optimized biological effect of the same.

In fact, it should be noted that the dye in accordance with the disclosure herein is allowed to be locally released with a homogeneous spreading exactly in the place subjected to the endoscopic diagnosis. For example, as disclosed herein, the dye is released in the colon, including also the right part of the colon.

Thanks to the specific oral solid composition and to the defined administration schedule disclosed herein, the dye orally administered is locally released and also completely absorbed in the intestinal tract, such as in the colon and further such as in the right part of the colon. In that way, that which is disclosed herein avoids any undesired early release or early absorption in anatomical tracts such as the stomach or small intestine not of interest in the endoscopic diagnosis.

The localized absorption of the dye on the intestinal mucosa allows the dye to penetrate in the cells wherein it is retained leading to an improved staining effect, increased contrast and better detection and the related diagnosis.

Improved absorption of the dye is of particular relevance when methylene blue is used as the dye for the endoscopic diagnosis. That follows because methylene blue is a “vital dye” able to be uptaken by the cells in a different way than by the extracellular space.

Moreover, oral administration of the composition defined herein according to the administration schedule disclosed herein can lead to detection of a larger number of lesions in the smaller size category, thus improving the endoscopic diagnosis.

The solid composition disclosed herein, administered orally as disclosed herein, advantageously can further extensively stain the colonic mucosas, reducing colonoscopy subjectivity due to the endoscopist or operator involved in the endoscopic diagnosis, and consequently improving efficacy of the diagnostic evaluation itself.

The oral composition disclosed herein also can reduce the time involved in the endoscopic diagnosis by avoiding the dead times involved with spraying the dye and then washing it out from the mucosa to be examined.

The examples below also clarify the oral composition and administration schedule disclosed herein, without entailing any restrictions whatsoever with respect thereto.

### EXAMPLES

### Example 1: Controlled-Release Coated Tablet for Endoscopy (Colon)

| | | |
|---|---|---|
| Description | UOM | Amt. per tablet |

Components

| | | |
|---|---|---|
| Carmine indigo | mg | 50.0 |
| Lecithin | mg | 5.0 |
| Stearic acid | mg | 10.0 |
| Mannitol | mg | 100.0 |
| Lactose | mg | 50.0 |
| Hydroxyethyl cellulose | mg | 25.0 |
| Sodium starch glycolate | mg | 6.0 |
| Colloidal hydrated silica | mg | 3.0 |
| Magnesium stearate | mg | 2.0 |

Coating

| | | |
|---|---|---|
| Methacrylic acid copolymer type A (Eudragit L) | mg | 6.0 |
| Methacrylic acid copolymer type B (Eudragit S) | mg | 6.0 |
| Triethyl citrate | mg | 1.2 |
| talc | mg | 5.8 |
| Titanium dioxide | mg | 3.0 |

The applied process provides for mixing the dye with the lecithin surfactant, stearic acid, mannitol and half of the required amount of magnesium stearate. After compacting the mixture, followed by granulation, then cellulose, sodium starch glycolate, colloidal silica and the remaining magnesium stearate are added and, after further mixing, the final compression is then carried out to obtain 250 mg tablets. The tablet is then coated with a mixture of methacrylic copolymers of type A and B, so as to extend the resistance to dissolution in vitro up to a pH ≥7, characteristic of the ileocecal and colon environment.

### Example 2: Controlled-Release Release Coated Tablet for Endoscopy (Colon)

| | | |
|---|---|---|
| Description | UOM | Amt. per tablet |

Components

| | | |
|---|---|---|
| Methylene blue | mg | 50.0 |
| Lecithin | mg | 5.0 |
| Stearic acid | mg | 10.0 |
| Mannitol | mg | 100.0 |
| Dibasic Sodium phosphate | mg | 25.0 |
| Hydroxypropyl methylcellulose | Mg | 35.0 |
| Sodium starch glycolate | mg | 6.0 |
| Colloidal hydrated silica | mg | 2.0 |
| Magnesium stearate | mg | 2.0 |

Coating

| | | |
|---|---|---|
| Methacrylic acid copolymer type A (Eudragit L) | mg | 6.0 |
| Methacrylic acid copolymer type B (Eudragit S) | mg | 6.0 |
| Triethyl citrate | mg | 1.2 |
| talc | mg | 5.8 |
| Titanium dioxide | mg | 3.0 |

The preparation process provides for mixing the dye with lecithin, stearic acid and dibasic sodium phosphate, compaction thereof into wafers followed by dry granulation, mixing with the remaining components of the nucleus and the final compression to the weight of 235 mg/tablet. The coating uses methacrylic derivatives as base and an alcohol solvent to facilitate the application phase.

The tablets thus obtained were subjected to dissolution test in vitro, revealing a good resistance to the acid environment and a progressive transfer of the dye in the neutral environment having a pH of 7.2.

### Example 3: Controlled Release Coated Tablet for Endoscopy (Colon)

| | | |
|---|---|---|
| Description | UOM | Amt. per tablet |

Components

| | | |
|---|---|---|
| Methylene blue | mg | 200.0 |
| Lecithin | mg | 5.0 |
| Stearic acid | mg | 14.0 |
| Methylhydroxypropyl cellulose | mg | 180.0 |
| Mannitol | mg | 140.0 |
| Microcrystalline cellulose | mg | 140.0 |
| talc | mg | 10.0 |
| Colloidal hydrated silica | mg | 5.0 |
| Magnesium stearate | mg | 6.0 |

Coating

| | | |
|---|---|---|
| Methacrylic acid copolymer type A (Eudragit L) | mg | 16.0 |
| Methacrylic acid copolymer type B (Eudragit S) | mg | 16.0 |
| Triethyl citrate | mg | 6.4 |
| talc | mg | 15.6 |
| Titanium dioxide | mg | 6.0 |

The composition is obtained through advance mixing and granulation of the dye, the lecithin as the amphiphilic component, the stearic acid as a component of the lipophilic matrix, mannitol and part of the magnesium stearate. After screening the granules obtained preliminarily, the remaining components and in particular cellulose, capable of producing the hydrophilic matrix structure, are added. The final pharmaceutical form, obtained by compressing the mixture of powders and granules, and weighing about 720 mg, is subjected to coating with a mixture of copolymers of methacrylic derivatives of type A and B, supported by a plasticiser, i.e., triethyl citrate, by a dye pigment, i.e., titanium dioxide, and by an anti-stick agent, such as talc, using ethyl alcohol as a solvent.

The tablet thus obtained resists dissolution in vitro in buffers with pH<2 and allows a progressive release of the dye substances in buffers with pH >7 as here below detailed:
Dissolution % after 2 hours in pH 1 dissolution medium: 0% (spec≤10%)
Dissolution % after 4 hour in pH 7.2 dissolution medium: 27%
Dissolution % after 8 hour in pH 7.2 dissolution medium: 84% (spec≥80%)

The same tablets of this Example 3 have been used for a PK Phase 1 trial, where 200 and 400 mg single doses have been compared and where the following averaged values of the main PK parameters have been recorded:

for the 200 mg dose
mean t_{lag}≥3 hours
mean tₘₐₓ (hours) 16.10±4.01
bioavailability compared to injected dose (F_{abs} %): 139.19±52.0
mean Cₘₐₓ (ng/ml) 1662.2±501.93
urine excretion (mean % of the dose)=39.67±19.19
mean t_{1/2} (hours) 20.19±4.68,
   whereas for the 400 mg dose the main parameters recorded have been:
mean t_{lag}≥3 hours
mean tₘₐₓ (hours) 17.67±3.60
mean Cₘₐₓ (ng/ml) 1635.67±729.57
urine excretion (mean % of the dose)=22.99±14.92
mean t_{1/2} (hours) 17.25±7.43

### Example 4: Controlled-Release Coated Tablet for Endoscopy (Colon)

| | | |
|---|---|---|
| Description | UOM | Amt. per tablet |

Tablet

| | | |
|---|---|---|
| Indigo Carmine | mg | 100.0 |
| Sodium Lauryl sulphate | mg | 3.0 |
| Stearic acid | mg | 12.0 |
| Lactose | mg | 130.0 |
| Microcrystalline cellulose | mg | 80.0 |
| Sodium starch glycolate | mg | 10.0 |
| Colloidal hydrated silica | mg | 12.0 |
| Magnesium stearate | mg | 3.0 |

Coating

| | | |
|---|---|---|
| Methacrylic acid copolymer type A | mg | 10.0 |
| Methacrylic acid copolymer type B | mg | 10.0 |
| Triethylcitrate | mg | 8.0 |
| Talc | mg | 6.0 |
| Titanium dioxide | mg | 3.8 |

The process provides for mixing the components of layer 1 and compression thereof, followed by the compression of a mixture of powders and granules obtained from a previous compaction of some components of the layer 2, precisely the dye, lecithin, stearic acid, the microcrystalline cellulose and mannitol with half of the magnesium stearate, with the remaining co-formulants.

The tablet, weighing about 250 mg, has two differently coloured distinct layers formulated for differentially releasing the dye both in the gastric sector and in the subsequent intestinal sector.

### Example 5: Controlled-Release Coated Tablet for Endoscopy (Colon)

| Description | UOM | Amt. per tablet |
|---|---|---|
| Methylene blue | mg | 25.0 |
| Lecithin | mg | 3.0 |
| Stearic acid | mg | 10.0 |
| Methylhydroxypropyl cellulose | mg | 90.0 |
| Mannitol | mg | 121.0 |
| Microcrystalline cellulose | mg | 60.0 |
| talc | mg | 3.0 |
| Colloidal hydrated silica | mg | 5.0 |
| Magnesium stearate | mg | 3.0 |

Coating

| | | |
|---|---|---|
| Methacrylic acid copolymer type A (Eudragit L) | mg | 8.0 |
| Methacrylic acid copolymer type B (Eudragit S) | mg | 8.0 |
| Triethyl citrate | mg | 3.2 |
| talc | mg | 7.8 |
| Titanium dioxide | mg | 3.0 |

The composition is obtained through ordered mixing of the dye, the lecithin as the amphiphilic component, the stearic acid as a component of the lipophilic matrix; then the remaining components were added and in particular the celluloses, capable of producing the hydrophilic matrix structure up to completion of the formula. The final pharmaceutical form, obtained by compressing the mixture of powders and granules, unitary weighing of about 320 mg, is subjected to coating with a mixture of copolymers of methacrylic derivatives of type A and B, supported by a plasticiser, triethyl citrate, by a dye pigment, titanium dioxide, and by an anti-sticking agent, such as talc, using ethyl alcohol or water or mixtures thereof as solvent.

The tablet thus obtained revealed in vitro a substantial non-dissolution (<10%) at pH 1 for 2 hours and a progressive dissolution in a simulated intestinal medium with pH 7.2 with a release of:
about 10% after 1 hour (with specification limit ≤30%)
about 44% after 4 hours and
more than 90% at the eighth hour (with specification limit ≥80%).

The tablets have been used also to determine in human volunteers, subjected to a standard bowel cleansing procedure through the administration of a 4-liters, PEG containing bowel preparation solution (commercially known as Selg® Esse 1000), the PK characteristics of 2 doses of Methylene Blue administered as divided doses individually containing 25 mg of the dye.

The same tablets have been used for a PK Phase I trial, where 100 and 200 mg single doses have been compared and where the following averaged values of the main PK parameters have been recorded:

for the 100 mg dose
mean t_{lag}≥3 hours
mean tₘₐₓ (hours) 12.0 (individual values 9-16)
mean Cₘₐₓ (ng/ml) 573.60±175.83
urine cumulative excretion (mean % of the dose) in 0-60 hours=28.02±11.71
mean t_{1/2} (hours) 13.87±5.09
   whereas for the 200 mg dose the main parameters recorded have been
mean t_{lag}≥3 hours
mean tₘₐₓ (hours) 16.0 (individual values 10-24)
mean Cₘₐₓ (ng/ml) 1149.12±261.95
urine cumulative excretion (mean % of the dose) in 0-60 hours=38.67±15.8
mean t_{1/2} (hours) 15.08±5.85

In order to optimize the way to administer the tablets as a function of the mucosal staining results, a clinical trial has been carried out with the above described tablets, using as a discriminating parameter a scoring system (TSC) originally created and composed of a number between 0 and 20, calculated as the sum of each individual staining score ranging 0 to 5 (where 0 is not stained at all, 1 is “traces”, i.e. poor dye traces in colonic mucosa, 2 “detectable”, i.e. relevant to a staining of at least 25% of the area, 3 is “acceptable”, i.e. relevant to a staining of at least 50% of the area, 4 is “good”, i.e. relevant to a staining of at least 75% of the area, and 5 is “overstained”, i.e. relevant to an overstaining not enabling an endoscopist to see the mucosal surface with the due accuracy in the 100% of the area), measured in the 4 segments of the colonic tract and indicated as right or ascending colon, transverse colon, descending colon and sigma-rectum; this scoring system was used to select the most reliable administration schedule of the dye with the aim of optimizing the tablets administration and the lesions detection possibilities during the colonoscopy procedure.

So, using the tablets formulated as described, the administration schedules has been changed on small groups of patients and the corresponding staining score has been determined. Since the importance of the colonic mucosal staining is that a well stained aspect should be extended to all the colonic segments, not only focused in a single colonic district, an additional parameter has been taken into account: the NSA or Number of Stained Area with staining score ≥2. With the application of these two parameters (TSC and NSA) the determination of the tablets administration schedule in order to obtain the best conditions for the endoscopist to enhance the detection of all the lesions in the colonic mucosa, has been carried out.

In the table below the different administration schedules of the two doses tested are reported with the corresponding measured staining score:

A) for 150 mg dose,
with the administration schedule A including 2 tablets (tbs.) before drinking the bowel prep, 2 tbs. after the first litre (L), 2 tbs. after the second L and the mean staining score was 6.8±4.0 and the mean stained colonic segments (NSA) was 1.3.
with the administration schedule B including 6 tablets (tbs.) before drinking the bowel prep, the mean staining score was 2.3±2. 4 and the mean stained colonic segments (NSA) was 0.4.
with the administration schedule C including 6 tablets (tbs.) at the end of the bowel prep, the mean staining score was 8.1±3. 6 and the mean stained colonic segments (NSA) was 1.5.
   B) for 200 mg dose,
with the administration schedule D including 4 tablets (tbs.) before drinking the bowel prep, 2 tbs. after the first L, 2 tbs. after the second L and the mean staining score was 7.0±5.0 and the mean stained colonic segments (NSA) was 1.3.
with the administration schedule E including 8 tablets (tbs.) at the end of bowel preparation solution the mean staining score was 9.8±4.4 and the mean stained colonic segments (NSA) was 2.3.
with the administration schedule F including 2 tablets (tbs.) before drinking the bowel prep, 2 tbs. after the first L, 2 tbs. after the second L and 2 tbs. at the end of bowel preparation the mean staining score was 9.3±4.1 and the mean stained colonic segments (NSA) was 2.2.
with the administration schedule G including 2 tablets (tbs.) before drinking the bowel prep, 2 tbs. after the first L, 2 tbs. after the second L and 2 tbs. at the end of bowel preparation the mean staining score (TSC) was 10.5±7.8 and the mean stained colonic segments (NSA) was 1.5.
with the administration schedule H including 4 tbs. after the third L, and 4 tbs. at the end of bowel preparation the mean staining score (TSC) was 10.0±3.2 and the mean stained colonic segments (NSA) was 2.1.
with the administration schedule I including 4 tbs. after the second L and 4 tbs. after the third L of bowel preparation the mean staining score (TSC) was 11.4±3.8 and the mean stained colonic segments (NSA) was 2.8.
with the administration schedule J including 2 tablets (tbs.) after the second L 3 tbs: after the third L and 3 tbs. at the end of bowel preparation the mean staining score (TSC) was 11.6±3.5 and the mean stained colonic segments (NSA) was 2.6.

Using the same tablets described in Example 5, with a total dose of 200 mg of methylene blue and an administration schedule of 2 tbs. after the second L, 3 after the third L and 3 at the end of bowel preparation, two Phase II clinical trials have been carried out: A) on 96 completed patients for cancer screening and surveillance, and B) an additional 52 patients belonging to a high risk population, i.e. the patients with long standing ulcerative colitis.

A) The cancer screening and surveillance trial had the aim of evaluating the polyp and adenoma detection rate in patients undergoing a full colonoscopy after colonic mucosal staining obtained with Methylene Blue MMX® tablets. Therefore, the primary end-point was to evaluate the polyp detection rate and the adenoma detection rate after colonic mucosal staining,

Other Secondary end-point(s) have been set, precisely:
to classify polyps and adenomas detected after colonic mucosal staining,
to evaluate the serrated lesion detection rate.
to evaluate the mucosal staining efficacy of Methylene Blue MMX® tablets
the Bowel cleansing quality was also evaluated according to the validated Boston Bowel Preparation Scale (BBPS).
to collect data about safety and tolerability of Methylene Blue MMX® tablets after administration of a single dose of 200 mg.

The subjects started the tablets intake in the afternoon before the colonoscopy day and had to drink at least 250 mL of preparation every 15 min, so that the bowel preparation intake could be completed 4 h after.

Measured trial variables:
Frequency of patients with polyps.
Frequency of patients with adenomas.
Number of adenomas in the right colon for each patient.
Number of detected serrated lesions for each patient.
Mucosal staining score for each area; total staining score.
Boston bowel preparation score for bowel cleansing preparation quality.
Time to reach the caecum.
Time to withdrawal from caecum to exit.
Adverse events.
Vital signs (blood pressure, heart rate, saturation in peripheral blood), body weight.

The obtained results are here below summarized.
1) Mucosal abnormalities (polyps, adenomas and serrated lesions) in each colonic region per patient (A) and as total number (B)

| | Methylene blue MMX ® tablets | | | | | |
|---|---|---|---|---|---|---|
| | Number of | | Number of | | Number of | |
| Colonic region | polyps | | adenomas | | serrated lesions | |
| (A) | | | | | | |
| All regions | 1.8 ± 2.9 | 1.0 (0-20) | 0.9 ± 1.7 | 0 (0-14) | 0.7 ± 1.8 | 0 (0-10) |
| Right colon | 0.6 ± 1.2 | 0 (0-9) | 0.4 ± 1.1 | 0 (0-8) | 0.1 ± 0.4 | 0 (0-2) |
| Caecum | 0.2 ± 0.5 | 0 (0-3) | 0.2 ± 0.4 | 0 (0-3) | 0 ± 0.2 | 0 (0-2) |
| Ascending | 0.3 ± 0.6 | 0 (0-3) | 0.2 ± 0.6 | 0 (0-3) | 0.1 ± 0.3 | 0 (0-2) |
| colon | | | | | | |
| Hepatic flexure | 0.2 ± 0.6 | 0 (0-5) | 0.1 ± 0.5 | 0 (0-4) | 0 ± 0.1 | 0 (0-1) |
| Transverse | 0.1 ± 0.4 | 0 (0-2) | 0.1 ± 0.3 | 0 (0-1) | 0 ± 0.2 | 0 (0-1) |
| colon | | | | | | |
| Splenic flexure | 0.1 ± 0.3 | 0 (0-2) | 0.1 ± 0.3 | 0 (0-2) | 0 ± 0 | 0 (0-0) |
| Descending | 0.1 ± 0.3 | 0 (0-1) | 0.1 ± 0.2 | 0 (0-1) | 0 ± 0.2 | 0 (0-1) |
| colon | | | | | | |
| Sigmoid | 0.4 ± 0.8 | 0 (0-4) | 0.1 ± 0.4 | 0 (0-2) | 0.2 ± 0.6 | 0 (0-3) |
| Rectum | 0.5 ± 1.6 | 0 (0-10) | 0.1 ± 0.6 | 0 (0-5) | 0.4 ± 1.3 | 0 (0-9) |

| | | | | | | |
|---|---|---|---|---|---|---|
| (B) | | | | | | |
| All regions | 61 | (63.5) | 45 | (46.9) | 26 | (27.1) |
| Right colon | 32 | (33.3) | 24 | (25.0) | 9 | (9.4) |
| Caecum | 14 | (14.6) | 13 | (13.5) | 2 | (2.1) |
| Ascending | 16 | (16.7) | 10 | (10.4) | 5 | (5.2) |
| colon | | | | | | |
| Hepatic flexure | 9 | (9.4) | 7 | (7.3) | 2 | (2.1) |
| Transverse | 12 | (12.5) | 8 | (8.3) | 4 | (4.2) |
| colon | | | | | | |
| Splenic flexure | 6 | (6.3) | 5 | (5.2) | 0 | (0.0) |
| Descending | 7 | (7.3) | 4 | (4.2) | 3 | (3.1) |
| colon | | | | | | |
| Sigmoid | 21 | (21.9) | 12 | (12.5) | 8 | (8.3) |
| Rectum | 19 | (19.8) | 9 | (9.4) | 12 | (12.5) |

All endoscopic findings were classified by a histopathologist. The detected lesions were predominantly low grade tubular adenomas, hyperplastic serrated lesions, low grade serrated adenomas, low grade tubular-villous adenomas but also high grade adenomas with carcinoma in situ, including tubular-villous, villous and tubular lesions. The mucosal staining efficacy of Methylene Blue MMX® tablets was on average “acceptable” with the 50% of the mucosa stained in all 4 examined colonic regions. Bowel cleansing quality was on average “good” according to the total BBPS score.

### Conclusions:

The polyp detection rate and the adenoma detection rate/patient in the whole colon were on average 1.8±2.9 detected polyps and 0.9±1.7 detected adenomas. The polyp detection rate ranged from 0 to 20 polyps per subject and was higher in the rectum with a maximum of 10 polyps and in the right colon with a maximum of 9 lesions. The adenoma detection rate ranged from 0 to 14 adenomas per subject and was higher in the rectum with a maximum of 5 adenomas. In the right colon, the maximum detection rate was 8 detected adenomas. Serrated lesions ranged from 0 to 10, with the highest prevalence in the rectum with a maximum of 9 lesions.

As summarized in the following table, polyps were detected at a frequency of 64%, adenomas at a frequency of 47% and serrated lesions at a frequency of 27.1% (9% of subjects in the right colon, considered at the same severity level than adenomas).

| Number of patients | Number of patients | Number of patients |
|---|---|---|
| with polyps (%) | with adenomas (%) | with serrated (%) |
| 61 (63.5) | 45 (46.9) | 26 (27.1) |

There was a good consistency between the pit pattern scores and histological classification.

The most frequently affected region for polyps was sigmoid and rectum (21.9% and 19.8% respectively) and serrated lesions most frequently in the rectum (12.5%). Considering the 3 areas right, transverse and descending colon, the transverse colon is that with the lowest detection rate, followed by right and descending colon.

The analysis was performed also by subdividing the intraepithelial neoplasiae by size. The rate of detection by lesion size is summarised in the following table. The number of detected polyps, adenomas and serrated lesions <5 mm; mean (±SD) and median (range) are reported.

| | Methylene blue MMX ® tablets | | |
|---|---|---|---|
| | Number of | Number of | Number of |
| Lesion size | polyps | adenomas | serrated lesions |
| ≤5 mm | 1.3 ± 2.3 | 0.5 ± 1.1 | 0.6 ± 1.7 |

Smaller lesions (≤5 mm) were predominant in frequency, and that is remarkable inasmuch that the conventional white light colonoscopy, such smaller lesions are the most difficult to detect. Polyps ≤5 mm had a maximum number of 15 detected abnormalities. The maximum number of detected adenomas ≤5 mm was 9 and 10 for the serrated lesions ≤5 mm.

The proportion of subjects with detected polyps by size, with detected adenomas and with detected serrated lesions are presented also in the following summary table. The proportion of subjects with detected polyps, adenomas and serrated lesions by colonic region; number (%) of subjects is reported.

| | | Methylene blue MMX ® tablets | | |
|---|---|---|---|---|
| | | | | Subjects with |
| | | Subjects with | Subjects with | at least one |
| Popu- | Lesion | at least one | at least one | serrated |
| lation | size | polyp n (%) | adenoma n (%) | lesion n (%) |
| FAS | ≤5 mm | 50 (52.1) | 30 (31.3) | 23 (24.0) |
| (N = 96) | 6-9 mm | 12 (12.5) | 10 (10.4) | 3 (3.1) |
| | ≥10 mm | 24 (25.0) | 22 (22.9) | 3 (3.1) |

### Conclusions:

Efficacy of Methylene Blue MMX® 25 mg modified release tablets was investigated and proven in the detection of the mucosal lesions in all the colonic districts, particularly with the lesions <5 mm. A large proportion of patients, compared to data in the literature, has been found affected by the presence of polyps and adenomas, particularly in the sigmoid-rectum district and also in the right colon.

B) The efficacy of Methylene Blue MMX® 25 mg modified release tablets was investigated in patients with ulcerative colitis with a diagnosis of ≥8 years and colitis activity index<8, This population was chosen because patients with long standing ulcerative colitis have a significantly higher risk for the development of colitis associated colorectal cancers.

The intraepithelial neoplasia detection rate was 16% (8 out of 50 subjects belonging to PP population) with a total of 10 intraepithelial neoplasiae detected in the 8 subjects. Intraepithelial neoplasiae were most frequently found in the rectum-sigma segment (RES), followed by descending colon (DC) and transverse colon (TC) at the same frequency, and finally by the ascending colon (AC). The number of intraepithelial neoplasiae/subject was 0.2±0.5.

As summarized below, false positive findings represented 8% (4 out of 50 subjects), whilst the false negative findings were 6% (3 out of 50). The method had a sensitivity greater than 50% (precisely 57.1%) and a specificity greater that 90% (precisely 90.7%.)

Study results are consistent with the higher range of the literature data obtained with the chromo-endoscopy technology spray of the dye instead of the oral administration of the dye during bowel preparation as disclosed herein. The dye spray technology was able to dramatically reduce the time of examination compared to the random biopsies: in the cited spray chromo-endoscopy trial, intraepithelial neoplasiae were detected at a rate of 15.48% in the same population, with a solution of 0.1% methylene blue sprayed using a catheter.

Detection rate of intraepithelial neoplasiae and true and false positive and negative findings analysis population (N=52).

| Proportion of | | | | |
|---|---|---|---|---|
| subjects with | True | False | True | False |
| intraepithelial | positive | positive | negative | negative |
| neoplasiae | findings | findings | findings | findings |
| 8 (15.4) | 4 (7.7) | 4 (7.7) | 41 (78.8) | 3 (5.8) |

The mucosal staining efficacy of Methylene Blue MMX® tablets was confirmed on average “acceptable” with 50% of the mucosa stained in all 4 examined colonic regions, with the best stained colonic segment resulting in the ascending colon, the region where it's more difficult to find the dysplastic lesions. The majority of subjects had NSA in all 4 regions. Bowel cleansing quality was on average “good” according to the total BBPS score.

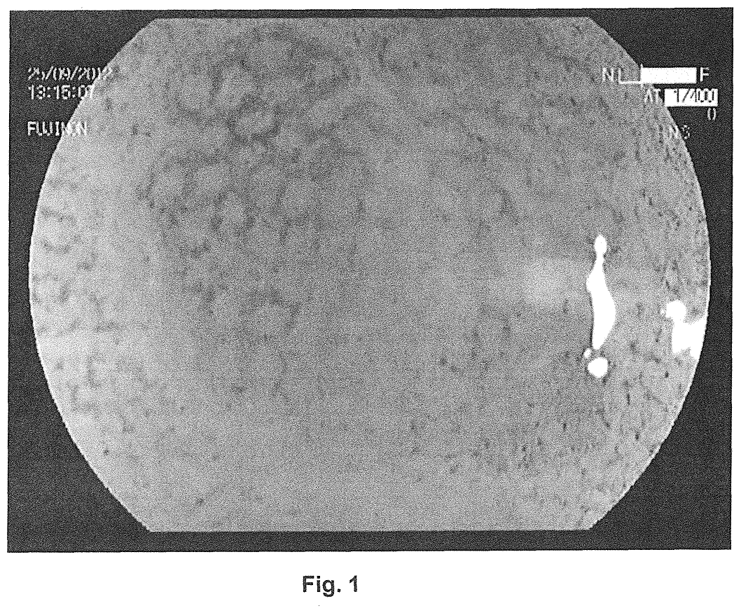

Two images of colon endoscopy are below reported to also better clarify the invention. FIG. 1 shows the contrast enhancing efficacy of the dye according to the present invention in perceiving the deep mucosal tissue structure, with the foci of the glands well defined and darkened in a pre-polyp alteration of the colonic mucosa.

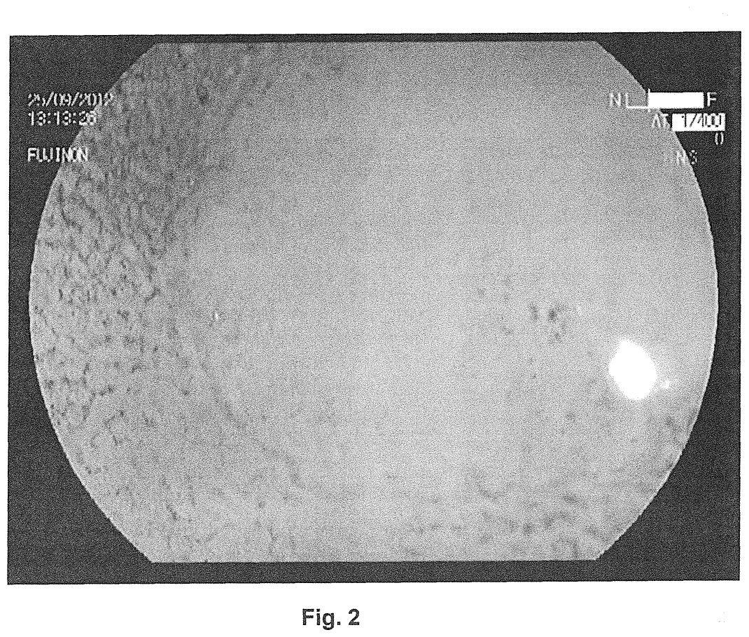

FIG. 2 shows the semi-continuous blue line defines exactly the borders of the colonic flat lesion that the endoscopist has to take out, allowing a better resolution of the lesion intervention and extraction. The tissue definition is absolutely enhanced owing to the orally administered dye as disclosed herein. With the conventional spraying techniques, the same performance cannot be obtained since little time is available between spray and observation (seconds or a couple of minutes).

### Example 6: Methylene Blue (MB) Tablet and Placebo Tablet for Phase III Clinical Study

A. Methylene Blue Tablet Used in Phase III Clinical Study

| | |
|---|---|
| | Amount/Tablet |
| Components | (mg) |

Tablet Core

| | |
|---|---|
| Methylthioninium Chloride (Methylene Blue) | 25.0 |
| (as anhydrous substance) | |
| Stearic Acid | 10.0 |
| Lecithin | 3.0 |
| Microcrystalline cellulose | 60.0 |
| Hydroxypropylmethylcellulose | 90.0 |
| Mannitol | 121.0 |
| Talc | 3.0 |
| Silica, Colloidal Anhydrous | 5.0 |
| Magnesium Stearate | 3.0 |

Coating

| | |
|---|---|
| Methacrylic acid copolymer type A | 8.0 |
| Methacrylic acid copolymer type B | 8.0 |
| Talc | 7.8 |
| Titanium Dioxide | 3.0 |
| Triethylcitrate | 3.2 |

* Mannitol should compensate the Methylthioninium chloride (Methylene blue) water content and purity.

The composition is obtained through ordered mixing of the dye, the lecithin as amphiphilic component, the stearic acid as a component of the lipophilic matrix; then the remaining components were added and in particular the celluloses, capable of producing the hydrophilic matrix structure up to completion of the formula. The final pharmaceutical form, obtained by compressing the mixture of powders and granules, unitary weighing of about 320 mg, is subjected to coating with a mixture of copolymers of methacrylic derivatives of type A and B, supported by a plasticiser, triethyl citrate, by a dye pigment, titanium dioxide, and by an anti-sticking agent, such as talc, using ethyl alcohol or water or mixtures thereof as solvent. The final film coated tablet has a theoretical weight of about 350 mg containing an active ingredient Methylene blue) quantity equivalent to 25 mg of dried substance.

B. Placebo Tablet Used in Phase III Clinical Study

| | |
|---|---|
| | Amount/Tablet |
| Components | (mg) |

Tablet Core

| | |
|---|---|
| Stearic Acid | 10.0 |
| Lecithin | 3.0 |
| Microcrystalline cellulose | 85.0 |
| Hydroxypropylmethylcellulose | 90.0 |
| Mannitol | 121.0* |
| Talc | 3.0 |
| Silica, Colloidal Anhydrous | 5.0 |
| Magnesium Stearate | 3.0 |

Coating

| | |
|---|---|
| Methacrylic acid copolymer type A (Eudragit ® L) | 8.0 |
| Methacrylic acid copolymer type B (Eudragit ® S) | 8.0 |
| Talc | 7.8 |
| Titanium Dioxide | 3.0 |
| Triethylcitrate | 3.2 |

Tablets are prepared in a similar manner as for the methylene blue tablets.

### Example 7: Phase III Clinical Trial

The methylene blue (MB) tablets of Example 6 (also simply referred to as “MB tablets” in this example) were studied in in a multicenter, double-blind, randomized, placebo-controlled phase III clinical study in subjects undergoing screening or surveillance colonoscopy. The objective of the study was the evaluation of the Adenoma or Carcinoma detection rate in patients undergoing a full colonoscopy after mucosal staining obtained with the MB tablets of Example 6 compared to placebo tablets of Example 6 (also simply referred to as “placebo tablets” in this example). The detection rate was defined as the proportion of patients with at least one histologically proven Adenoma or Carcinoma.

The study was conducted as a placebo controlled trial: in fact, for one group of patients, placebo tablets according to Example 6 were administered, and the patients underwent the colonoscopy in the same conditions as the patients administered with the MB tablets of Example 6. In this way, due to the lack of the dye in the placebo tablets, an immediate comparison between the Adenoma or Carcinoma detection rate in patients with unstained colon (patients administered with placebo tablets) and in patients with colon stained with a methylene blue (patients administered with MB tablets) was obtained. In other words, in the study the standard of care (white light endoscopy) and the chromoendoscopy by oral administration of the MB tablets were compared. In the acquisition and recording of the colonoscopies, the latest and more technologically advanced, high definition (HD) endoscopes were used. Thus, the study allowed the direct comparison of the effect of the MB tablets on the adenoma or carcinoma detection rate to the current gold standard colonoscopy, i.e. the high definition (HD) white light colonoscopy. For this reason, in the context of the present example, the terms “placebo”, “white light high definition endoscopy” (WLHD) and “white light endoscopy” can be used interchangeably, and definitively indicate the current standard of care for the endoscopic examination of the colon. Due to the unavoidable impossibility to obtain double-blinding for the clinical study, since the endoscopist performing the colonoscopy was able to see if the colon was blue colored or unstained, an additional, underpowered, masking group was introduced. The patients of the masking group received a reduced dose of MB tablets of Example 6 (100 mg vs 200 mg of the standard dose). These patients had the colon stained in blue, but were not taken into account in the calculation of the study parameters. In this way, while performing the colonoscopy, the endoscopist was not aware if a patient belonged to the group of the full dose (whose patients were part of the statistical calculation) or to the “masking” group (whose patients received a lower dose of the composition and were not part of the statistical analysis).

### Objective

The objective of the study was the evaluation of the histologically proven Adenoma or Carcinoma detection rate in patients undergoing a full colonoscopy with and without mucosal contrast enhancement, obtained through the administration of the MB tablets of Example 6 up to a total dose of 200 mg of methylene Blue. The lack of mucosal contrast, obtained with placebo tablets was equivalent to a standard white light colonoscopy endoscopic procedure, the current standard of care.

### Study Subjects

Subjects aged between 50 and 75 years undergoing full colonoscopy for screening or surveillance of colorectal cancer were recruited.

### Diet, Bowel Cleansing Preparation and Dose Regimen

Diet: In preparation for the colonoscopy patients adopted a low residue diet for three days prior to the colonoscopy. On the third day of the low residue diet patients must fast for at least 3 hours before starting intake of the bowel prep and study drug.

Bowel cleansing preparation: all subjects received a full dose regimen of 4 liters PEG-based bowel cleansing preparation starting in the late afternoon (after 6 pm) before the colonoscopy day. The subjects drank at least 250 mL of solution every 15 min, so that the intake of the cleansing preparation, and study drug were completed in 4 hours.

Dose regimen: The subjects were randomized 2:2:1 into three groups.

Group One (Methylene blue full dose—200 mg) received 8 MB tablets of Example 6 (with a total dose of 200 mg of methylene blue): 3 MB tablets (75 mg of methylene blue) after the first 2 liters of bowel preparation, 3 MB tablets (75 mg of methylene blue) after a total of 3 liters of bowel preparation and, finally, 2 MB tablets (50 mg of methylene blue) after a total of 4 liters of bowel preparation had been consumed.

Group Two (Placebo) received an oral dose of placebo tablets of Example 6 identical to Group 1 with respect to the number of tablets and the intake schedule: 3 placebo tablets after the first 2 liters of bowel preparation, 3 placebo tablets after a total of 3 liters of bowel preparation and, finally, 2 placebo tablets after a total of 4 liters of bowel preparation had been consumed.

Group Three (Methylene blue low dose—100 mg) was included only for masking purposes in order to reduce the acquisition bias due to the lack of investigator and subject blinding between placebo and the Methylene Blue (MB) tablet 200 mg groups. This unpowered masking group was treated with the MB tablets of Example 6 up to a total dose of 100 mg of methylene blue (4 MB tablets i.e., half the dose of methylene blue with respect to Group 1). In order to maintain the number of tablets unchanged with respect to the Groups 1 and 2 (whose subjects received 8 tablets in total), 4 placebo tablets were administered in addition to the methylene blue tablets: 1 MB tablet (25 mg of methylene blue) and two placebo tablets after the first 2 liters of bowel preparation, additional 2 MB tablets (50 mg of methylene blue) and one placebo tablet after a total of 3 liters of bowel preparation, and, finally, 1 MB tablet (25 mg of methylene blue), and one placebo tablet after a total of 4 liters of bowel preparation solution had been consumed.

All the patients participating to the study, regardless of whether they belonged to Group one, Group two or Group three, received a timetable detailing the volumes of bowel cleansing preparation to be consumed, and the times to be respected:

| Time from | | Number of tablets of |
|---|---|---|
| consumption of | Volume of bowel | solid composition |
| first volume | cleansing solution | comprising 25 mg of |
| of bowel cleansing | (mL) to be consumed | methylene blue to be |
| solution (minutes) | by the human | administered to the human |
| 0 | 250 | 0 |
| 15 | 250 | 0 |
| 30 | 250 | 0 |
| 45 | 250 | 0 |
| 60 | 250 | 0 |
| 75 | 250 | 0 |
| 90 | 250 | 0 |
| 105 | 250 | 0 |
| 120 | 250 | 3 |
| 135 | 250 | 0 |
| 150 | 250 | 0 |
| 165 | 250 | 0 |
| 180 | 250 | 3 |
| 195 | 250 | 0 |
| 210 | 250 | 0 |
| 225 | 250 | 0 |
| 240 | Consume water | 2 |

The subjects had to drink about 250 mL of bowel cleansing preparation every 15 minutes, equivalent to 1 liter of bowel cleansing preparation every hour. The timeframe between an oral administration of tablets and the following one had to be 1 hour.

### Study Endpoints

Primary end-point: the primary end-point of this study was to assess the detection efficacy of chromoendoscopy performed with Methylene Blue 25 mg tablets according to Example 6 versus placebo tablets according to Example 6 (white light endoscopy) in terms of the proportion of subjects with at least one histologically proven Adenoma or Carcinoma (i.e. Adenoma Detection Rate). Adenoma was defined as a histologically proven Vienna Grade 3 to 4.2 or a histologically proven traditional serrated adenoma (TSA), or a histologically proven sessile serrated adenoma (SSA). Histologically proven Carcinoma was defined as Vienna Grade 4.3 to 5b.

Secondary End-Points:
False positive rate between treatment and placebo control arms; the rate being defined as the proportion of patients with no histologically confirmed Adenoma or Carcinoma within any of the subjects excised lesions and the subject having undergone at least one excision.

### Study Schedule

Screening Visit 01: during the screening visit, the patients underwent a blood sampling to check renal and hepatic function. Females of childbearing potential underwent a serum pregnancy test.

Randomization Visit 01A: during the visit, the investigator verified if patients' blood results met eligibility criteria and, if so, he assigned the study medication and randomized the patients. The investigator instructed the patients on the recommended diet for the days leading up to colonoscopy, and bowel cleansing preparation as per instructions.

In preparation for the colonoscopy patients adopted a low residue diet for three days prior to the colonoscopy.

Day before colonoscopy: patients self-administered the investigational product, consisting of methylene blue tablets of Example 6 (Group One: treatment arm) or matching placebo tablets of Example 6 (Group Two: white light endoscopy arm) or both (Group Three: reduced dose arm), at home during the intake of the bowel cleansing preparation according to the given instructions.

Day of colonoscopy 02: patients returned to the clinic for the colonoscopy. The colonoscopy was performed using high definition (HD) colonoscope. Narrow-Band-Imaging (NBI) and all other electronic chromoendoscopy and contrast enhancement techniques, as well as zoom endoscopy or magnification were not permitted. The endoscopist performed the full colonoscopy and recorded and removed all the detected adenoma and/or carcinoma. The endoscopist recorded the morphology and size and classified the found polyps, adenomas and carcinomas, and recorded the pit pattern according to the Paris and Kudo's classification and following the endoscopy charter.

The criteria for removal were any abnormal area that without magnification had any of the following three elements (1) obvious elevation or depression, (2) mucosal nodular irregularity, (3) interruption of the course of superficial vascular network. All adenoma and carcinoma were removed with standard techniques of polyp resection. Whenever the adenoma or carcinoma could not be removed because of their size or morphology, several biopsies were taken for histopathological evaluation. Each endoscopy was recorded on digital media. After conclusion of the endoscopic examination, blood samples were taken for the assessment of the patients' liver and renal function, and the subjects were allowed to leave the clinic.

Follow-up visit 03: A follow-up visit was scheduled within 3-7 days from colonoscopy. Additional blood tests were taken for confirmation and to assess recovery.

### Histopathological Assessment

Tissue bioptic specimens collected and fixed in formalin were shipped to the histopathology laboratory of the local sites, where the slides were prepared for shipment to the central histopathologists. The histological diagnosis performed at the local histopathology laboratory was provided to the patient and to the physician in order to correctly manage the patient. An additional section was taken from each paraffin block, stained, mounted and shipped from the local laboratory to a central laboratory for the trial analysis. The central histopathologist graded all Adenomas and Carcinomas removed according to the adapted revised Vienna classification) and serrated classification: in particular, the central histopathologist graded all traditional serrated adenoma or sessile serrated adenomas. The central laboratory histopathologist provided the microscopic assessment which was considered for the study endpoints.

Grades 3-5 of the modified Vienna criteria and histologically proven traditional serrated adenoma (TSA), and histologically proven sessile serrated adenoma (SSA) of the serrated lesions classification, were included as histological evidence of Adenoma or Carcinoma.

### Vienna Criteria for Gastrointestinal Neoplasia

| Vienna category | Description |
|---|---|
| 1 | Negative for Neoplasia/Dysplasia |
| 2 | Indefinite for Neoplasia/Dysplasia |
| 3 | Non-invasive low grade Neoplasia |
| | (Low grade adenoma/Dysplasia) |
| 4.1 | Mucosal high grade Neoplasia |
| 4.2 | High grade Adenoma/Dysplasia |
| 4.3 | Non-invasive Carcinoma (Carcinoma in situ) |
| 4.4 | Suspicion of invasive Carcinoma |
| 5.a | Intramucosal Cacinoma |
| 5.b | Submucosal Carcinoma or beyond |

### The Serrated Lesions Classification

| Category | Description |
|---|---|
| SSA | Sessile serrated adenomas |
| TSA | Traditional serrated adenomas |
| HP | Hyperplastic polyps |
| FP | Fibroblastic polyps |
| MP | Mixed polyps |

### Endoscopic Procedure Central Reading

Mucosal surface pit pattern and nature of lesions were measured and recorded electronically in vivo, in real time, during the endoscopy. A second reading of the recorded video was performed by the central reviewer. The central reviewer gave an opinion on the lesions resected, as to the need for excision, lack of excision, and whether the excision was taken from a stained or not stained area.

### Data Analysis

The primary analysis was a logistic regression on the proportion of patients with at least one histologically proven Adenoma or Carcinoma found during colonoscopy. Treatment, center, age, sex, reason for colonoscopy (screening, surveillance within 2 years from previous colonoscopy, and surveillance after more than 2 years from previous colonoscopy) and number of excisions (categorized as being “≤3”, “4-6” and “>6”) were included in the regression model as fixed effects.

The other secondary end-points were summarized by descriptive statistics. The false positive rate was evaluated as the proportion of subjects who will not have histologically confirmed Adenoma or Carcinoma but will have at least one excision.

### Analysis Set

At the end of the trial, a total of 1346 subjects were enrolled; among them, 97 subjects were excluded for being screening failures. The remaining were subdivided in the following analysis sets:

Full Analysis Set (FAS): all randomized subjects who received at least one dose of the investigational medicinal product and underwent colonoscopy (regardless of the completion status). This analysis set was used for the primary efficacy analysis. The FAS population comprised 1205 subjects, subdivided as follows: 485 subjects in the methylene blue full dose group, 479 subjects in the placebo group and 241 subjects in the methylene blue low dose group.

Per Protocol Set (PP): all randomized subjects who fulfilled the study protocol requirements with no major deviations that may affect study results. This analysis set was used for sensitivity analyses. The PP population comprised 1137 subjects, subdivided as follows: 455 subjects in the methylene blue full dose group, 457 subjects in the placebo group and 225 subjects in the methylene blue low dose group.

### Study Results

At the end of the study, the results reported in the following tables were obtained.

TABLE 1
Proportion of subjects with at least one histologically proven Adenoma or Carcinoma (Full analysis set).

| | | Methylene | Methylene | |
|---|---|---|---|---|
| | | blue low | blue full | |
| | Placebo | dose (100 mg) | dose (200 mg) | |
| | (N = 479) | (N = 241) | (N = 485) | |
| | n (%) | n (%) | n (%) | |
| Subjects with at least one histologically proven Adenoma or | 229 (47.81) | 124 (51.45) | 273 | (56.29) |
| Carcinoma (i.e.: Adenoma Detection Rate) | | | | |

| Odds Ratio and 95% CI of Methylene Blue Full Dose vs. Placebo | 1.41 | [1.09, 1.81] |
|---|---|---|
| Difference in Proportions and 95% CI of Methylene Blue Full Dose vs. Placebo | 8.48 | [2.20, 14.77] |
| p-value of Fisher's exact test of Methylene Blue Full Dose vs. Placebo | 0.0099 | |
| Odds Ratio and 95% CI of Methylene Blue Low Dose vs. Placebo | 1.16 | [0.85, 1.58] |
| Difference in Proportions and 95% CI of Methylene Low Dose vs. Placebo | 3.64 | [−4.09, 11.38] |
| p-value of Fisher's exact test of Methylene Blue Low Dose vs. Placebo | 0.3851 | |
| Odds Ratio and 95% CI of Methylene Blue Full Dose vs. Methylene Blue Low Dose | 1.22 | [0.89, 1.66] |
| Difference in Proportions and 95% CI of Methylene Full Dose vs. Methylene Blue Low Dose | 4.84 | [−2.86, 12.54] |

| | |
|---|---|
| p-value of Fisher's exact test of Methylene Full Dose vs. Methylene Blue Low Dose | 0.2353 |

TABLE 2
Proportion of subjects with at least one histologically proven Adenoma or Carcinoma (Per Protocol).

| | | Methylene | Methylene | |
|---|---|---|---|---|
| | | blue low | blue full | |
| | Placebo | dose (100 mg) | dose (200 mg) | |
| | (N = 457) | (N = 225) | (N = 455) | |
| | n (%) | n (%) | n (%) | |
| Subjects with at least one histologically proven Adenoma or | 219 (47.92) | 121 (53.78) | 265 | (58.24) |
| Carcinoma (i.e.: Adenoma Detection Rate) | | | | |

| Odds Ratio and 95% CI of Methylene Blue Full Dose vs. Placebo | 1.52 | [1.17, 1.97] |
|---|---|---|
| Difference in Proportions and 95% CI of Methylene Blue Full Dose vs. Placebo | 10.32 | [3.88, 16.76] |
| p-value of Fisher's exact test of Methylene Blue Full Dose vs. Placebo | 0.0018 | |
| Odds Ratio and 95% CI of Methylene Blue Low Dose vs. Placebo | 1.26 | [0.92, 1.74] |
| Difference in Proportions and 95% CI of Methylene Low Dose vs. Placebo | 5.86 | [−2.11, 13.82] |
| p-value of Fisher's exact test of Methylene Blue Low Dose vs. Placebo | 0.1663 | |
| Odds Ratio and 95% CI of Methylene Blue Full Dose vs. Methylene Blue Low Dose | 1.20 | [0.87, 1.65] |
| Difference in Proportions and 95% CI of Methylene Full Dose vs. Methylene Blue Low Dose | 4.46 | [−3.47, 12.40] |

| | |
|---|---|
| p-value of Fisher's exact test of Methylene Full Dose vs. Methylene Blue Low Dose | 0.2854 |

TABLE 3
Proportion of subjects with at least one histologically proven Adenoma or Carcinoma (i.e.:
Adenoma Detection Rate), subdivided by the number of excisions (Full analysis set).

| | | Methylene | Methylene | |
|---|---|---|---|---|
| | | blue low | blue full | |
| | Placebo | dose (100 mg) | dose (200 mg) | |
| | n (%) | n (%) | n (%) | |
| Number of excisions 0-1 | 50 (18.94) | 25 (20.83) | 61 | (26.18) |
| Odds Ratio and 95% CI of Methylene Blue Full Dose vs. Placebo: | | | 1.52 | [0.99, 2.32] |
| p-value of Fisher's exact test of Methylene Blue Full Dose vs. Placebo: | | | 7.24 | [−0.12, 14.60] |
| Difference in Proportions and 95% CI of Methylene Blue Full Dose vs. Placebo: | | | 0.0663 | |
| Number of excisions ≤3 | 135 (35.90) | 69 (38.12) | 164 | (45.30) |
| Odds Ratio and 95% CI of Methylene Blue Full Dose vs. Placebo: | | | 1.48 | [1.10, 1.99] |
| p-value of Fisher's exact test of Methylene Blue Full Dose vs. Placebo: | | | 9.40 | [2.34, 16.46] |
| Difference in Proportions and 95% CI of Methylene Blue Full Dose vs. Placebo: | | | 0.0107 | |
| Number of excisions 4-6 | 61 (88.41) | 36 (87.80) | 74 | (85.06) |
| Odds Ratio and 95% CI of Methylene Blue Full Dose vs. Placebo: | | | 0.75 | [0.29, 1.92] |
| p-value of Fisher's exact test of Methylene Blue Full Dose vs. Placebo: | | | −3.35 | [−13.99, 7.29] |
| Difference in Proportions and 95% CI of Methylene Blue Full Dose vs. Placebo: | | | 0.6399 | |
| Number of excisions >6 | 33 (97.06) | 19 (100.00) | 35 | (97.22) |

| | | |
|---|---|---|
| Odds Ratio and 95% CI of Methylene Blue Full Dose vs. Placebo: | 1.06 | [0.06, 17.66] |
| p-value of Fisher's exact test of Methylene Blue Full Dose vs. Placebo: | 0.16 | [−7.65, 7.98] |

| | |
|---|---|
| Difference in Proportions and 95% CI of Methylene Blue Full Dose vs. Placebo: | 1.0000 |

TABLE 4
False Positive Rate (Full analysis set).

| | | Methylene | Methylene | |
|---|---|---|---|---|
| | | blue low | blue full | |
| | Placebo | dose (100 mg) | dose (200 mg) | |
| | (N = 479) | (N = 241) | (N = 485) | |
| | n (%) | n (%) | n (%) | |
| Subjects with excision | 326 (68.06) | 168 (69.71) | 356 | (73.40) |
| Subjects with excisions and without any | 97 (29.75) | 44 (26.19) | 83 | (23.31) |
| histologically proven Adenoma or Carcinoma | | | | |

| Difference in False Positive Rates and 95% CI of Methylene Blue Full Dose vs. Placebo | −6.44 | [−13.07, 0.19] |
|---|---|---|
| p-value of difference in False Positive Rates | <0.0001 | |
| Difference in False Positive Rates and 95% CI of Methylene Blue Low Dose vs. Placebo | −3.56 | [−11.86, 4.73] |
| p-value of difference in False Positive Rates | <0.0001 | |
| Difference in False Positive Rates and 95% CI of Methylene Blue Full Dose vs. Methylene | −2.88 | [−10.84, 5.09] |
| Blue Low Dose | | |

| | |
|---|---|
| p-value of difference in False Positive Rates | <0.0001 |

TABLE 5
False Positive Rate (Per Protocol).

| | | Methylene | Methylene | |
|---|---|---|---|---|
| | | blue low | blue full | |
| | Placebo | dose (100 mg) | dose (200 mg) | |
| | (N = 457) | (N = 225) | (N = 455) | |
| | n (%) | n (%) | n (%) | |
| Subjects with excision | 314 (68.71) | 163 (72.44) | 343 | (75.38) |
| Subjects with excisions and without any | 95 (30.25) | 42 (25.77) | 78 | (22.74) |
| histologically proven Adenoma or Carcinoma | | | | |

| Difference in False Positive Rates and 95% CI of Methylene Blue Full Dose vs. Placebo | −7.51 | [−14.26, −0.77] |
|---|---|---|
| p-value of difference in False Positive Rates | <0.0001 | |
| Difference in False Positive Rates and 95% CI of Methylene Blue Low Dose vs. Placebo | −4.49 | [−12.91, 3.93] |
| p-value of difference in False Positive Rates | <0.0001 | |
| Difference in False Positive Rates and 95% CI of Methylene Blue Full Dose vs. Methylene | −3.03 | [−11.07, 5.02] |
| Blue Low Dose | | |

| | |
|---|---|
| p-value of difference in False Positive Rates | <0.0001 |

TABLE 6
Proportion of subjects with at least one histologically proven Adenoma or Carcinoma
(Full analysis set) - Logistic regression model.

| | | | | | | Adjusted odds | |
|---|---|---|---|---|---|---|---|
| | Type 3 analysis of effects | | | | | ratio | |
| | Degree | Wald | | | | | 95% |
| | of | Chi- | | | Comparison | Point | Wald |
| Effect | Freedom | Square | p-value | Comparison | p-value | estimate | CI |
| Treatment | 1 | 6.3255 | 0.0119 | Methylene blue full dose | 0.0119 | 1.45 | [1.09, 1.94] |
| Analysis Centre | 18 | 24.3675 | 0.1434 | vs placebo | | | |
| Age | 1 | 6.1448 | 0.0132 | | | | |
| Sex | 1 | 18.3570 | <0.0001 | | | | |
| Reason for Colonscopy | 2 | 5.0276 | 0.0810 | | | | |
| Number of Excisions | 2 | 97.4150 | <0.0001 | | | | |

### Discussion

The study allowed to directly compare the effect of the MB tablets of Example 6 on the adenoma or carcinoma detection rate to the current gold standard colonoscopy, i.e. the white light colonoscopy. The placebo tablets of Example 6 were used for blinding purposes, and were given to patients of the control group; the subjects of this group, having the colon unstained (due to the lack of the dye in the placebo tablets), represent subjects undergoing the endoscopic examination of the colon with the current standard of care, i.e. the white light colonoscopy. In order to maintain both the subjects and the endoscopist blind, a “masking” group was added: the subjects of this group were administered with a low dose (100 mg instead of 200 mg) of methylene blue.

The high quality of the clinical study was assured by several measures:
Each endoscopist had to satisfy the requirements of an Endoscopy Charter with a tailor-made admission test;
Each colonoscopy has been recorded with a new high definition system and reviewed in remote & blind mode by a Central Reader (5 in total) to prevent bias;
Each lesion has been analyzed by the histological lab of the site and re-analyzed in blind by a Central Histological Reader (2 in total) to avoid bias and confirm diagnosis according to an agreed Histology Charter specifying lesions classification.

The methylene blue at a dose of 200 mg (administered in form of the 25 mg tablets of Example 6, a total of 8 tablets per subject) showed a statistically significant improvement of the adenoma detection rate (proportion of subjects with at least one proven adenoma or carcinoma) with respect to the placebo tablets, i.e., the standard of care (white light HD endoscopy—WLHD). As a matter of fact, the adenoma detection rate of methylene blue full dose was 56.29% vs 47.81% of the placebo (WLHD) in the FAS population: in other words, the use of the MB tablets of Example 6 allowed to obtain an increase of 17.7% in adenoma detection rate (ADR) with respect to the standard of care, as shown in Table 7.

TABLE 7
Adenoma Detection Rates (defined as proportion of subjects with
at least one proven adenoma or carcinoma) comparison between
methylene full dose (200 mg) and placebo (corresponding to the
standard of care, i.e. WLHD) - (Full Analysis Set).

| | | Methylene |
|---|---|---|
| | Placebo | blue full |
| | (WLHD) | dose (200 mg) |
| | (N = 479) | (N = 485) |
| Adenoma detection rate (ADR) | 47.81% | 56.29% |

| | |
|---|---|
| Absolute difference in ADR between | 8.48% |
| methylene blue full dose and placebo | |
| Percent increase in ADR for methylene | 17.7% |
| full dose vs placebo | |
| p-value | 0.0099 |
| Odds ratio | 1.41 |

These results are even better when calculated in the PP population: the PP population, in fact, represent a subset of subjects who completed the study without major deviations (such as, by way of example, lack of compliance to the investigational tablets, full colonoscopy not fully executed, lack of adequate bowel cleansing, etc.). The PP therefore represents a subset that shows the real effects of the full dose of 200 mg of methyelene blue when the study protocol procedures are strictly followed. In other words, this subset shows the real efficacy of the study drug. In this subset, the adenoma detection rate of methylene blue full dose was 58.24% vs 47.92% of the placebo (WLHD): in other words, the use of the MB tablets of Example 6 allowed to obtain an increase of 21.5% in adenoma detection rate (ADR) with respect to the standard of care, as shown in Table 8.

TABLE 8
Adenoma Detection Rates (defined as proportion of subjects
with at least one proven adenoma or carcinoma) comparison
between methylene full dose (200 mg) and placebo (corresponding
to the standard of care, i.e. WLHD) - (Per Protocol).

| | | Methylene |
|---|---|---|
| | Placebo | blue full |
| | (WLHD) | dose (200 mg) |
| | (N = 457) | (N = 455) |
| | (%) | (%) |
| Adenoma detection rate (ADR) | 47.92% | 58.24% |

| | |
|---|---|
| Absolute difference in ADR between | 10.32% |
| methylene blue full dose and placebo | |
| Percent increase in ADR for methylene | 21.5% |
| blue full dose vs placebo | |
| p-value | 0.0018 |
| Odds ratio | 1.52 |

What is really important is that such increase in ADR for the methylene blue full dose vs placebo was not accompanied by an increase of the False Positive Rate (FPR). On the contrary, both in the FAS and in the PP subsets, the FPR of methylene blue full dose was significantly lower than the FPR of the placebo (i.e. of WLHD): 23.31% for methylene blue full dose vs 29.75% for placebo (FAS) (4=6.44%) and 22.74% for methylene blue full dose vs 30.25% for placebo (PP) (4=6.44%). This means that methylene blue full dose resulted in a decrease of 21.6% (FAS) and of 24.8% (PP) in FPR compared to placebo (i.e. WLHD). The FPR demonstrates that the higher adenoma detection rate in the methylene blue full dose group vs placebo was not conditioned by the higher number of lesions resected (the higher the number of resected lesions, the higher the probability of finding an adenoma or carcinoma), but was due to the capacity of methylene blue to “flag” the colonic lesions and to make them more easily recognizable by the endoscopist. In other words, thanks to the formulation of the present invention, the endoscopists were able to identify (and, therefore, remove) more adenomatous or cancerous lesions compared to the current standard of care (WLHD). The comparison between the FPR for methylene blue full dose and placebo are reported in Table 9 (FAS) and 10 (PP).

TABLE 9
Comparison between False Positive Rates of methylene
full dose (200 mg) and placebo (corresponding to
the standard of care, i.e. WLHD) - (FAS).

| | | Methylene |
|---|---|---|
| | Placebo | blue full |
| | (WLHD) | dose (200 mg) |
| | (N = 479) | (N = 485) |
| | n | n |
| Total number of patients with excisions | 326 | 356 |
| Number of patients with excisions, but | 97 | 83 |
| without an adenoma or carcinoma | | |
| False Positive Rate | 29.75% | 23.31% |

| | |
|---|---|
| Absolute difference in FPR between | −6.44% |
| methylene blue full dose and placebo | |
| Percent decrease in FPR for methylene | 21.6% |
| full dose vs placebo | |
| p-value | <0.0001 |

TABLE 10
Comparison between False Positive Rates of methylene
full dose (200 mg) and placebo (corresponding to
the standard of care, i.e. WLHD) - (PP).

| | | Methylene |
|---|---|---|
| | Placebo | blue full |
| | (WLHD) | dose (200 mg) |
| | (N = 457) | (N = 455) |
| Total number of patients with excisions | 314 | 343 |
| Number of patients with excisions, but | 95 | 78 |
| without an adenoma or carcinoma | | |
| False Positive Rate | 30.25% | 22.74% |

| | |
|---|---|
| Absolute difference in FPR between | −7.51% |
| methylene blue full dose and placebo | |
| Percent decrease in FPR for methylene | 24.8% |
| full dose vs placebo | |
| p-value | <0.0001 |

The effect of methylene blue was more evident in the patients with a low number of removed lesions. In fact, it is well known that, for patients with a high number of lesions, the probability of finding a precancerous (adenoma) or cancerous lesion is higher than in patients with a lower number of lesions. The lower the number of excisions, the higher the difficulty that a precancerous lesion can be detected in a patient. This was evident also in the present study, where the effect of methylene blue on ADR, compared to placebo, was more evident in the subsets of subjects with a number of excisions ≤3 both in the FAS and in the PP, as evident from Tables 11 and 12.

TABLE 11
Comparison between ADR in patients
with 0-1 or ≤3 excisions (FAS).

| | | Methylene |
|---|---|---|
| | Placebo | blue full |
| | (WLHD) | dose (200 mg) |
| | (N = 479) | (N = 485) |
| | (%) | (%) |
| ADR in patients with number of excisions | 18.94% | 26.18% |
| 0-1 | | |
| Absolute difference in ADR between | 7.24% | |
| methylene blue full dose and placebo | | |
| Percent increase in ADR for methylene full | 38.2% | |
| dose vs placebo | | |
| p-value | 0.0663 | |
| ADR in patients with number of | 35.90% | 45.30% |
| excisions ≤3 | | |

| | |
|---|---|
| Absolute difference in ADR between | 9.40% |
| methylene blue full dose and placebo | |
| Percent increase in ADR for methylene full | 26.2% |
| dose vs placebo | |
| p-value | 0.0107 |

TABLE 12
Comparison between ADR in patients
with 0-1 or ≤3 excisions (PP).

| | | Methylene |
|---|---|---|
| | Placebo | blue full |
| | (WLHD) | dose (200 mg) |
| | (N = 479) | (N = 485) |
| ADR in patients with number of excisions | 17.42% | 25.32% |
| 0-1 | | |
| Absolute difference in ADR between | 9.15% | |
| methylene blue full dose and placebo | | |
| Percent increase in ADR for methylene full | 45.4% | |
| dose vs placebo | | |
| p-value | 0.0256 | |
| ADR in patients with number of | 48.48% | 68.24% |
| excisions ≤3 | | |

| | |
|---|---|
| Absolute difference in ADR between | 11.47% |
| methylene blue full dose and placebo | |
| Percent increase in ADR for methylene full | 40.8% |
| dose vs placebo | |
| p-value | 0.00026 |

The logistic regression model analyzed the impact of each one of the key parameters on the whole statistical significance of the trial results. The point estimate and the limits calculated with this model are key indicators of the trial results. The logistic regression model confirmed that the higher adenoma detection rate obtained with methylene blue full dose compared to placebo (WLHD) was due to the efficacy of the treatment and not to external factors, such as the clinical center where the study was performed.

In an additional analysis (see Table 13), the adenoma detection rate in subjects with histologically proven adenomas only (thus, excluding the subjects with carcinomas) were calculated: the data showed that the methylene blue full dose has an adenoma detection rate on the subset of subjects with histologically proven adenoma of 55.26% compared to 45.93% of the placebo (FAS population). This means that methylene blue full dose resulted in a percent increase in adenoma detection rate in subjects with adenoma only of 20.3% compared to placebo (Table 13). Considering that the adenomas are recognized as precursors of colorectal cancer, it is evident that methylene blue 200 mg increases the capacity of the endoscopist to see and remove these precursors, and thus to prevent their transformation in advanced cancer.

TABLE 13
Proportion of subjects with histologically
proven adenoma (excluding carcinoma) - FAS.

| | | Methylene |
|---|---|---|
| | Placebo | blue full |
| | (WLHD) | dose (200 mg) |
| | (N = 479) | (N = 485) |
| Total number of subjects with a | 220 | 268 |
| histologically proven adenoma | | |
| Percentage of subjects with a histologically | 45.93% | 55.26% |
| proven adenoma | | |

| Absolute difference in proportion of subjects | 9.33% |
|---|---|
| with a histologically proven adenoma | |
| between methylene blue full dose and | |
| placebo | |
| Percent increase in proportion of subjects | 20.3% |
| with a histologically proven adenoma for | |
| methylene blue full dose vs placebo | |
| p-value | 0.046 |
| Odds ratio | 1.45 [1.13, 1.87] |

TABLE 14
Proportion of subjects with non-polypoid lesions - FAS.

| | | Methylene |
|---|---|---|
| | Placebo | blue full |
| | (WLHD) | dose (200 mg) |
| | (N = 479) | (N = 485) |
| Total number of subjects with non-polypoid | 168 | 213 |
| lesions | | |
| Percentage of subjects with non-polypoid | 35.09% | 43.92% |
| lesions | | |

| Absolute difference in proportion of subjects | 8.83% |
|---|---|
| with a histologically proven adenoma | |
| between methylene blue full dose and | |
| placebo | |
| Percent increase in proportion of subjects | 25.2% |
| with a histologically proven adenoma for | |
| methylene blue full dose vs placebo | |
| p-value | 0.0056 |
| Odds ratio | 1.45 [1.12, 1.88] |

TABLE 15
Proportion of subjects with diminutive adenomas - FAS.

| | | Methylene |
|---|---|---|
| | Placebo | blue full |
| | (WLHD) | dose (200 mg) |
| | (N = 479) | (N = 485) |
| Total number of subjects with diminutive | 148 | 180 |
| adenomas | | |
| Percentage of subjects with diminutive | 30.9% | 37.11% |
| adenomas | | |

| Absolute difference in proportion of subjects | 6.21% |
|---|---|
| with diminutive adenomas between | |
| methylene blue full dose and placebo | |
| Percent increase in proportion of subjects | 20.1% |
| with diminutive adenomas for methylene | |
| blue full dose vs placebo | |
| p-value | 0.0486 |
| Odds ratio | 1.32 [1.01, 1.72] |

It is worth noting that the methylene blue low dose group (100 mg of total dose), even though not powered to show any statistical significant differences from placebo and added to the clinical trial for masking purposes only, nonetheless showed adenoma detection rates and false positive rates intermediate between methylene blue full dose (200 mg) and placebo. This shows a dose response correlation between the dose of methylene blue and the ADR and the FPR.

The safety of tablets comprising methylene blue according to the embodiments disclosed herein was assessed in 1087 adults who received any dose of the tablets in conjunction with an oral bowel cleansing preparation prior to colonoscopy in 6 clinical trials. The median age of these subjects was 60 years (range, 21 to 80 years), and 58% were male. A total of 798 subjects received the full dose and formulation intended for commercialization (200 mg=8×25 mg tablets). Discontinuation of dosing due to an adverse event occurred in 0.5% of subjects receiving the 200 mg dose. The most common event leading to discontinuation of dosing was vomiting (0.4%). The primary safety database for the product is derived from a randomized, placebo-controlled trial (Study CB-17-01/06) in which 488 subjects received the tablets comprising the solid composition having a total dose of 200 mg of methylene blue. 241 subjects received a total dose of 100 mg of methylene blue, and 479 subjects received placebo in conjunction with an oral bowel cleansing preparation prior to colonoscopy.

The most common treatment emergent adverse reactions of any severity in Study CB-17-01/06 that occurred in at least 1% of subjects in the 200 mg dose group and with an incidence higher than in the placebo group are shown in Table 16.

TABLE 16
Treatment Emergent Adverse Reactions Occurring in ≥1%
of Subjects Receiving 200 mg methylene blue in Study
CB-17-01/06 with Incidence Greater Than Placebo

| | | |
|---|---|---|
| | 8 Tablets, each comprising | |
| | 25 mg methylene blue | |
| | (total dose = 200 mg | |
| | methylene blue) | Placebo |
| | (N = 488) | (N = 479) |
| Adverse Reaction | n (%) | n (%) |

| | | | |
|---|---|---|---|
| Chromaturia* | 234 | (48.0) | 7 (1.5) |
| Feces discolored* | 95 | (19.5) | 0 |
| Nausea | 29 | (5.9) | 17 (3.5) |
| Vomiting | 23 | (4.7) | 13 (2.7) |
| Headache | 13 | (2.7) | 8 (1.7) |
| Abdominal pain | 6 | (1.2) | 2 (0.4) |
| Hypotension | 5 | (1.0) | 3 (0.6) |

Less common adverse reactions (<1%) reported more frequently than placebo included: Renal and urinary disorders (Polyuria, dysuria); nervous system disorders (migraine); gastrointestinal disorders (abdominal discomfort, diarrhea, hematemesis); respiratory, thoracic and mediastinal disorders (cough); blood and lymphatic system disorders (anaemia); general disorders and administration site conditions (pain, chills); and eye disorders (blue scleral discoloration).

In some embodiments disclosed herein are delayed and extended-release solid compositions in the form of tablets, each containing 25 mg of methylene blue as dried substance. The tablets are coated with an enteric coating that is stable at acidic pH (in the stomach) but breaks down at or above pH 7, normally achieved in the terminal ileum. Once the film coating has dissolved, the extended-release formulation provides a slow release of the methylene blue dye, resulting in its homogeneous and prolonged dispersion on the surface of the colonic mucosa of a human to which the tablets are administered. Methylene blue stains the specialized columnar epithelium of intestine with high specificity and has been used to screen for colonic neoplasia, to diagnose villous atrophy, and to screen for areas of dysplasia and carcinoma. Abnormal staining is an excellent marker of dysplasia and/or early stage cancer. Methylene blue is a vital dye, which is absorbed by the epithelial cells of the intestine. In the gastrointestinal epithelium, the dysplastic epithelium areas and cancers have a different dye intake with respect to the surrounding healthy mucosa. After staining with methylene blue, these abnormalities appear as areas of altered staining or as a heterogeneous staining pattern against the surrounding mucosa. Due to the formulation of some of the solid compositions disclosed herein, the maximum local bioavailability of the methylene blue in the colon is achieved and, consequently, the contrast-enhancing effect is optimized.

In some embodiments, the delayed and extended tablet comprising methylene blue, is enteric coated with a polymer film, which breaks down at or above pH 7, allowing the release of methylene blue in the colon. The tablet core contains methylene blue with excipients that provide for extended release of the active ingredient throughout the whole length of the colon. Each tablet may also comprise stearic acid, lecithin, microcrystalline cellulose, hydroxypropylmethylcellulose, mannitol, colloidal silicon dioxide, magnesium stearate, methacrylic acid and methyl methacrylate copolymer (1:1), methacrylic acid and methyl methacrylate copolymer (1:2), triethylcitrate, talc, and titanium oxide.

Following the oral administration of the administration of the tablet of Example 6 at a total dose of 200 mg (8 extended-release tablets containing 25 mg each) in healthy subjects, peak plasma concentration (Cmax) was 1.15±0.26 μg/mL, with a median time to peak concentration (tmax) of 16.00 hours (10.00-24.00 hours), and an area under the curve (AUCO-co) of 28.56±9.76 μg/mL×h.

In a clinical study of the tablet of Example 6 at a dose of 200 mg, subjects excreted quantifiable amounts of unchanged methylene blue in urine through 60 hrs post-dose (last assessment). Cumulative excretion (Xu0-t) of unchanged methylene blue at 60 hours postdose was 77.34±31.61 mg, corresponding to 38.67±15.80% of the administered dose. In the same study, the mean half-life (t½) at the dose of 200 mg was determined to be approximately 15 hours since administration.

The efficacy of the tablet of Example 6 for the detection of adenoma or carcinoma in patients undergoing colonoscopy with high definition white light (HDWL) was evaluated in a multicenter, multinational, randomized, double-blind, placebo-controlled trial. Patients between 50 and 75 years of age scheduled for colonoscopy were randomized to a total dose of 200 mg of methylene blue, a total dose of 100 mg of methylene blue, or placebo. Patients self-administered the tablet of Example 6 and/or placebo during intake of the bowel cleansing preparation at home on the evening before the colonoscopy. A total of 1249 patients were randomized to the study. Overall, the median age was 62 years (range: 50-75 years), approximately 60% of the subjects were male, and more than 90% were White/Caucasian. The majority of patients were undergoing a colonoscopy either for screening (47.9%) or for surveillance after more than 2 years from previous colonoscopy (45.9%). The primary endpoint was the proportion of patients with at least one histologically proven Adenoma or Carcinoma detected. Histologically proven Adenoma was defined as Vienna Grade 3, 4.1, or 4.2, or a Traditional Serrated Adenoma (TSA), or Sessile Serrated Adenoma (SSA). Histologically proven Carcinoma was defined as Vienna Grade 4.3, 4.4, 5.a, or 5b. HDWL with a total dose of 200 mg of methylene blue using the tablets of Example 6 was superior to HDWL with placebo for the detection of adenoma or carcinoma (odds ratio [95% CI] of 1.41 [1.09, 1.81]; Fisher's exact test p value=0.0099).

In addition, the false positive rate (defined as the proportion of patients who had at least one lesion excised with no histologically confirmed adenoma or carcinoma within any of the excised lesions) for HDWL colonoscopy with the tablet of Example 6 was non-inferior to HDWL with placebo. The detection of difficult-to-detect lesions, such as non-polypoid (flat) lesions and small (<5 mm) lesions, was also higher with the tablet of Example 6 than with placebo. The results of the primary and selected secondary endpoints for are summarized in Table 17.

TABLE 17

| | HDWL + Tablets | | |
|---|---|---|---|
| | of Example 6 | HDWL + | |
| Endpoint | 200 mg | Placebo | Relative |
| PRIMARY EFFICACY ENDPOINT: | (N = 485) | (N = 479) | Improvement |
| Percent of subjects with at least one | 56.3% | 47.8% | 18% |
| histologically proven Adenoma or Carcinoma | | | |
| Odds Ratio versus Placebo [95% CI]¹ | 1.41 [1.09, 1.81]* | | |
| False Positives: Subjects with excisions | 23.3% | 29.8% | 22% |
| and without any histologically proven | | | |
| Adenoma or Carcinoma | | | |
| Difference from Placebo [95% CI]² | −6.44% [−13.07, 0.19]** | | |
| Subjects with at least one histologically | 55.3% | 45.9% | 20% |
| proven Adenoma without Carcinoma | | | |
| Odds Ratio versus Placebo [95% CI] | 1.45 [1.13, 1.87]* | | |
| Subjects with at least one non-polypoid | 43.9% | 35.1% | 25% |
| lesion | | | |
| Odds Ratio versus Placebo [95% CI] | 1.45 [1.12, 1.88]* | | |
| Subjects with at least one proven adenoma | 37.1% | 30.9% | 20% |
| or carcinoma <5 mm | | | |
| Odds Ratio versus Placebo [95% CI] | 1.32 [1.01, 1.72]* | | |

FPR = False positive rate;
HDWL = High Definition White Light Colonoscopy;
¹Fisher's exact test of Methylene Blue MMX 200 mg vs. Placebo
²Non-Inferiority Test. Null hypothesis to be rejected H0: FPRMethylene Blue 200 mg − FPRPlacebo ≥15%
*p < 0.05,
**p < 0.0001

The tablets comprising any of the compositions disclosed herein, including the tablet of Example 6 (extended release tablets 25 mg), may be supplied as off white to light blue, round, biconvex film coated tablets. The tablets comprising any of the compositions disclosed herein, including the tablet of Example 6, may be packaged in blister cards of 8 tablets contained in a cardboard carton. The tablets comprising any of the compositions disclosed herein, including the tablet of Example 6, may be stored at 20 to 25° C. (68 to 77° F.); with excursions permitted to 15° to 30° C. (59° to 86° F.) (See USP Controlled Room Temperature).

Humans administered the compositions disclosed herein, including the tablets of Example 6, may be instructed to discontinue administration of the tablets and seek immediate medical attention if any signs or symptoms of a hypersensitivity reaction occur: wheezing, difficulty breathing, difficulty of swallowing, skin reactions such as hives, rash or flushed skin, itching or tingling sensation, dizziness or light-headedness, weak pulse or rapid pulse, drop in blood pressure, seizure, or loss of consciousness. Female humans to which the compositions disclosed herein are administered, including the tablets of Example 6, may be instructed to tell their physician if they are pregnant or nursing.

Humans administered the compositions disclosed herein, including the tablets of Example 6, may be instructed to avoid driving and use of machines during treatment with the compositions as migraine, dizziness, presyncope, balance disorder, somnolence, confusion and disturbances in vision may occur. Humans administered the compositions disclosed herein, including the tablets of Example 6, may be instructed to take protective measures against exposure to light, because phototoxicity may occur after administration of compositions comprising methylene blue. Humans administered the compositions disclosed herein, including the tablets of Example 6, may be instructed to let their physician know if they have renal or hepatic disease. Humans administered the compositions disclosed herein, including the tablets of Example 6, may be instructed to take all 8 tablets as directed the evening before colonoscopy and to also complete the entire bowel preparation as directed by their physicians. Humans administered the compositions disclosed herein, including the tablets of Example 6, may be instructed should be swallowed whole with bowel preparation solution, water, or other clear liquid and not chewed, crushed or broken.

1. A method for improving the detection of pathologies in the colon, comprising orally administering to a human a bowel cleansing solution and 8 unit dosages of a solid composition, wherein the bowel cleansing solution and the 8 unit dosages of the solid composition are administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution;
c) 2 unit dosages of the solid composition after the intake of a 4^{th} liter of bowel cleaning solution;
wherein each unit dosage of the solid composition contains 25 mg of methylene blue, and wherein the method is characterized by one or more of the following:
i) an adenoma detection rate of at least about 40%,
ii) a false positive rate of not more than about 35%,
iii) detection rate of the proportion of subjects with non-polypoid lesion of at least about 30%, and
iv) detection rate of the proportion of subjects with diminutive adenoma of at least about 25%.

2. The method of claim 1, wherein method is characterized by an adenoma detection rate of at least about 40%, or at least about 45%, or at least about 50%, or at least about 55%.

3. The method of claim 2, wherein the adenoma detection rate is about 56.29%.

4. The method of claim 1, 2 or 3, wherein method is characterized by a false positive rate of not more than about 35%, or not more than about 30% or not more than about 25%.

5. The method of claim 4, wherein the false positive rate is about 22.74%.

6. The method of claim 1, wherein the method is characterized by a detection rate of the proportion of subjects with non-polypoid lesion of at least about 30%, or at least about 35%, or at least about 40%.

7. The method of claim 6, wherein the detection rate of the proportion of subjects with non-polypoid lesion is about 43.92%.

8. The method of claim 1 wherein the method is characterized by a detection rate of the proportion of subjects with diminutive adenoma of at least about 25%, or at least about 30%, or at least about 35%.

9. The method of claim 8, wherein the detection rate of the proportion of subjects with diminutive adenoma is about 37.11%.

10. The method of any one of claims 1-9, wherein each dosage unit of the solid composition comprises:
(a) 25 mg of methylene blue;
(b) at least one lipophilic compound;
(c) at least one hydrophilic compound;
(d) optionally at least one amphiphilic compound;
(e) optionally other physiologically acceptable excipients; and
(f) optionally a gastro-resistant coating.

11. The method of claim 10, wherein the at least one lipophilic compound has a melting point below 90° C.

12. The method of any one of claims 1-11, wherein the method enhances the colon mucosal lesion detection in the diagnosis of cancerous pathologies, precancerous pathologies, interval cancers, adenomas, carcinomas, serrated lesions, dysplasias, polyps, pseudopolyps, pre-polyps, hyperplastic lesions, and inflammatory pathologies.

13. A method of improving flagging mucosal lesions in the colon of a human comprising orally administering to a human one or more dosage units of a solid composition, wherein each dosage unit comprises:
(a) 25 mg of methylene blue;
(b) at least one lipophilic compound;
(c) at least one hydrophilic compound;
(d) optionally at least one amphiphilic compound;
(e) optionally other physiologically acceptable excipients; and
(f) optionally a gastro-resistant coating.

14. The method of claim 13, wherein the at least one lipophilic compound has a melting point below 90° C.

15. The method of claim 13 or 14, wherein the method further comprises orally administering a bowel cleansing solution to the human.

16. The method of claim 13, 14 or 15, wherein multiple dosages of the solid composition are administered to the human.

17. The method of claim 16, wherein the dosages are administered according to a schedule with respect to the administration of the bowel cleansing solution.

18. The method of claim 17, wherein 8 unit dosages of the solid composition are orally administered to the human according to the schedule comprising:
a) 3 unit dosages of the solid composition after the intake of 2 liters of bowel cleaning solution;
b) 3 unit dosages of the solid composition after the intake of 3 liters of bowel cleaning solution; and
c) 2 unit dosages of the solid composition after the intake of 4 liters of bowel cleaning solution.

19. The method of any one of claims 13-18, wherein the method enhances the colon mucosal lesion flagging in the diagnosis of cancerous pathologies, precancerous pathologies, interval cancers, adenomas, carcinomas, serrated lesions, dysplasias, polyps, pseudopolyps, pre-polyps, hyperplastic lesions, and inflammatory pathologies.

20. Solid composition containing at least one dye in association with at least one physiologically acceptable excipient which comprises:
(a) 25 mg of methylene blue;
(b) at least one lipophilic compound;
(c) at least one hydrophilic compound;
(d) optionally at least one amphiphilic compound;
(e) optionally other physiologically acceptable excipients; and
(f) optionally a gastro-resistant coating for use in improving the detection of pathologies in the colon, characterized in that 8 unit dosages thereof are orally administered to a human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution; and
c) 2 unit dosages of the solid composition after the intake of a 4^{th} liter of bowel cleaning solution,
wherein the improved detection of pathologies in the colon is characterized by one or more of the following:
i) an adenoma detection rate of at least about 40%,
ii) a false positive rate of not more than about 35%,
iii) detection rate of the proportion of subjects with non-polypoid lesion of at least about 30%, and
iv) detection rate of the proportion of subjects with diminutive adenoma of at least about 25%.

21. The solid composition of claim 20, wherein the at least one lipophilic compound has a melting point below 90° C.

22. The solid composition of claim 20 or 21, wherein method is characterized by an adenoma detection rate of at least about 40%, or at least about 45%, or at least about 50%, or at least about 55%.

23. The solid composition of claim 22, wherein the adenoma detection rate is about 56.29%.

24. The solid composition of any one of claims 20-23, wherein method is characterized by a false positive rate of not more than about 35%, or not more than about 30% or not more than about 25%.

25. The solid composition of claim 24, wherein the false positive rate is about 22.74%.

26. The solid composition of claim 20 or 21, wherein the method is characterized by a detection rate of the proportion of subjects with non-polypoid lesion of at least about 30%, or at least about 35%, or at least about 40%.

27. The solid composition of claim 26, wherein the detection rate of the proportion of subjects with non-polypoid lesion is about 43.92%.

28. The solid composition of claim 20 or 21, wherein the method is characterized by a detection rate of the proportion of subjects with diminutive adenoma of at least about 25%, or at least about 30%, or at least about 35%.

29. The solid composition of claim 28, wherein the detection rate of the proportion of subjects with diminutive adenoma is about 37.11%.

30. The solid composition of any one of claims 20-29, wherein the method enhances the colon mucosal lesion detection in the diagnosis of cancerous pathologies, precancerous pathologies, interval cancers, adenomas, carcinomas, serrated lesions, dysplasias, polyps, pseudopolyps, pre-polyps, hyperplastic lesions, and inflammatory pathologies.

31. Solid composition containing at least one dye in association with at least one physiologically acceptable excipient which comprises:
(a) 25 mg of methylene blue;
(b) at least one lipophilic compound;
(c) at least one hydrophilic compound;
(d) optionally at least one amphiphilic compound;
(e) optionally other physiologically acceptable excipients; and
(f) optionally a gastro-resistant coating
for use in improving the flagging of mucosal lesions in the colon of a human.

32. The solid composition of claim 31, wherein the at least one lipophilic compound has a melting point below 90° C.

33. The solid composition of claim 31 or 32, wherein multiple dosages of the solid composition are administered to the human.

34. The solid composition of claim 33, wherein the dosages are administered according to a schedule with respect to administration of the bowel cleansing solution.

35. The solid composition of claim 34, wherein 8 unit dosages of the solid composition are orally administered to the human according to the schedule comprising:
a) 3 unit dosages of the solid composition after the intake of 2 liters of bowel cleaning solution;
b) 3 unit dosages of the solid composition after the intake of a 3^{rd} liter of bowel cleaning solution; and
c) 2 unit dosages of the solid composition after the intake of a 4^{th} liter of bowel cleaning solution.

36. The solid composition of any one of claims 31-35, wherein the method enhances the colon mucosal lesion flagging in the diagnosis of cancerous pathologies, precancerous pathologies, interval cancers, adenomas, carcinomas, serrated lesions, dysplasias, polyps, pseudopolyps, pre-polyps, hyperplastic lesions, and inflammatory pathologies.

37. A method for improving the detection of pathologies in the colon of a human, comprising orally administering to the human 8 tablets of a solid composition and a volume of a bowel cleaning solution, wherein the solid composition is administered orally in three doses during the intake of the bowel cleansing solution according a schedule comprising:
(a) a first dose comprising administration of 3 tablets of the solid composition to the human following consumption of at least one liter of bowel cleansing solution;
(b) a second dose comprising administration of 3 tablets of the solid composition to the human about 1 hour following administration the first dose of the solid composition; and
(c) a third dose comprising administration of 2 tablets of the solid composition to the human about 1 hour following administration of the second dose of the solid composition.

38. The method according to claim 37, wherein at least 3 total liters of bowel cleansing solution is consumed by the human in combination with the administration of the 8 tablets of the solid composition.

39. The method according to claim 37 or 38, wherein the entire volume of bowel cleansing solution is consumed by the human in combination with the 8 tablets of the solid composition at least 8 hours prior to an endoscopic procedure being performed on the human.

40. The method of claim 37 or 38, wherein the human consumes one half or less of the total volume of bowel cleansing solution in combination with the administration of the 8 tablets of the solid composition the day before an endoscopic procedure is performed, and the remaining portion of the bowel preparation solution is consumed by the human on the day the endoscopic procedure is performed.

41. The method according to any one of claims 1 to 19 and 37 to 40, wherein the entire volume of bowel preparation solution is consumed at least two hours prior to the endoscopic procedure.

42. The method according to any one of claims 1 to 19 and 37 to 41, wherein, the bowel cleansing solution is consumed by the human according a schedule comprising: (a) the day before the endoscopic procedure, the human consumes a volume of at least 16 ounces of bowel preparation solution, followed by the consumption of at least 32 ounces of water over the next hour; and (b) the day of the endoscopic procedure, the human consumes at least 16 ounces of bowel preparation solution, followed by the consumption of at least 32 ounces of water over the next hour.

43. The method for improving the detection of pathologies in the colon of a human during an endoscopic procedure, comprising orally administering to the human 8 tablets of a solid composition and a volume of a bowel cleaning solution, wherein the solid composition is administered orally during the intake of the bowel cleansing solution according to a schedule comprising: (a) the day before the endoscopic procedure, the human consumes a volume of at least 16 ounces of bowel preparation solution, followed by the consumption of at least 32 ounces of water over the next hour; and (b) the day of the endoscopic procedure, the human consumes at least 16 ounces of bowel preparation solution, followed by the consumption of at least 32 ounces of water over the next hour.

44. The method according to claim 43, wherein the 8 tablets of the solid composition are administered to the human the day before the endoscopic procedure.

45. The method according to claim 43, wherein a portion of the 8 tablets of the solid composition are administered to the human the day before the endoscopic procedure, and the remaining tablets of the solid composition are administered to the human the day of the endoscopic procedure.

46. The method according to any one of claims 43 to 45, wherein the entire volume of bowel preparation solution is consumed by the human at least two hours prior to the performance of the endoscopic procedure.

47. The method according to any one of claims 43 to 46, wherein the 8 tablets of the solid composition are administered to the human at least 8 hours prior to the endoscopic procedure.

48. A method according to any one of claims 43 to 47, wherein the human is administered 8 tablets of a solid composition and consumes a volume of a bowel cleansing solution, wherein the bowel cleaning is consumed according to a schedule comprising: (a) the day before the endoscopic procedure, the human consumes a volume of at least 16 ounces of bowel preparation solution, followed by the consumption of at least 32 ounces of water over the next hour; and (b) the day of the endoscopic procedure, the human consumes at least 16 ounces of bowel preparation solution, followed by the consumption of at least 32 ounces of water over the next hour.

49. The method according to claim 43, wherein the 8 tablets of the solid composition are administered to the human and the entire volume of bowel cleansing solution is consumed by the human at least 8 hours prior to the endoscopic procedure.

50. The method according to claim 43, wherein the 8 tablets of the solid composition are administered to the human and the entire volume of bowel cleansing solution is consumed by the human at least 2 hours prior to the endoscopic procedure.

51. The method according to claim 43, wherein the 8 tablets of the solid composition are administered to the human at least 8 hours prior to the endoscopic procedure and the entire volume of the bowel cleansing solution is consumed by the human at least 2 hours prior to the endoscopic procedure.

52. The method according to any one of claims 37 to 51, wherein the human consumes a total volume of 4 liters of a bowel cleansing solution at a rate of 240 mL (8 ounces) every 10 minutes, until 4 the liters of the bowel cleansing solution are consumed or until the rectal effluent of the human is clear.

53. The method according to any one of claims 1 to 19 and 37 to 41, wherein the bowel cleansing solution is delivered to the human by nasogastric tube at a rate of from about 1.2 liters per hour to about 1.8 liters per hour.

54. The method according to any one of claims 1 to 19 and 37 to 41, wherein the human consumes a volume of bowel cleansing solution at a rate of 25 mL/kg/hour until 4 liters are consumed or until watery stool is clear and free of solid matter.

55. The method of any one of the claims 1 to 19, 37 and 43, wherein the solid composition is a modified release composition, an extended release composition, a delayed release composition or an extended and delayed release composition.

56. The method of any one of the claims 37 and 43, wherein the adenoma detection rate is of at least about 40% or at least about 45% or at least about 50% or at least about 55%.

57. The method of any one of the claims 37 and 43, wherein the adenoma detection rate is about 56.29%.

58. The method of any one of the claims 37 and 43, wherein the false positive rate is of not more than about 35% or not more than about 30% or not more than about 25%.

59. The method of any one of the claims 37 and 43, wherein the false positive rate is about 22.74%.
